(19) United States
(12) Patent Application Publication
(10) Pub. No.: US 2010/0081798 A1
(43) Pub. Date: Apr. 1, 2010

(54) METHOD FOR PRODUCING GLUCOSE BY ENZYMATIC HYDROLYSIS OF CELLULOSE THAT IS OBTAINED FROM MATERIAL CONTAINING LIGNO-CELLULOSE USING AN IONIC LIQUID THAT COMPRISES A POLYATOMIC ANION

(75) Inventors: Tim Balensiefer, Mannheim (DE); Hartwig Schroeder, Nussloch (DE); Stephan Freyer, Neustadt (DE); Giovanni D'Andola, Heidelberg (DE); Klemens Massonne, Bad Duerkheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/523,740

(22) PCT Filed: Jan. 22, 2008
(86) PCT No.: PCT/EP08/50710

§ 371 Date: Jul. 20, 2009

(30) Foreign Application Priority Data
Jan. 23, 2007 (EP) 07101034.2
Dec. 14, 2007 (EP) 07150040.9

(51) Int. Cl. C12P 19/02; C07H 3/02; C07G 1/00

(52) U.S. Cl. 530/500; 435/105; 536/1.11

(57) ABSTRACT

The present invention relates to a process for preparing glucose from a lignocellulose-comprising starting material, in which this is firstly treated with an ionic liquid and subsequently subjected to an enzymatic hydrolysis. The invention further relates to a process for preparing microbial metabolites, especially ethanol, in which the glucose obtained is additionally subjected to a fermentation.

The present invention relates to a process for preparing glucose and, if appropriate, further products of value, e.g. further sugars and/or lignin, from a lignocellulose-comprising starting material, in which this is firstly treated with an ionic liquid and subsequently subjected to an enzymatic hydrolysis. The invention further relates to a process for preparing microbial metabolites, especially ethanol, in which the glucose obtained is additionally subjected to a fermentation.

Cellulose is, at a share of about 700 billion metric tons of the estimated biomass stock of 1.5 trillion metric tons on earth, the most important representative in the group of organic biopolymers and a raw material which is used in a wide variety of ways. The hydrolysis of cellulose to glucose will gain particular importance in future, since this could open up, for example, a route to large amounts of bioethanol obtained by fermentation. However, cellulose is rarely present in pure or sufficiently concentrated form in the biomass available as raw material source, but is instead present essentially as a constituent of lignocellulose. The digestion and fractionation of lignocellulose into its main constituents of cellulose, lignin and hemicellulose are central objects of a still to be developed biorefinery concept which is to make effective and economical utilization of this renewable raw material possible. It is becoming increasingly clear that, in particular, the biofuel ethanol can only be prepared on a long-term basis if a cheaper process for degrading the cellulose present in the biomass is found. An increasing importance of glucose as intermediate in the chemical industry is also only conceivable if the raw materials basis is decoupled from the cultivation of starch- or sugar-comprising plants which at the present time serve mainly for food production.

A variety of methods of digesting lignocellulose as pretreatment for a subsequent enzymatic hydrolysis have been developed. In Appl. Microbiol. Biotechnol., 2002, 59, pp. 618-628, M. Galbe and G. Zacchi give an overview of the preparation of ethanol from lignocellulose sources. The conversion of lignocellulose into sugars and further into ethanol suffers from various problems. All known production processes comprise, as common step, the hydrolysis of the cellulose and, if appropriate, of the hemicellulose to the monomeric sugars. This hydrolysis can be carried out using concentrated acids, dilute acids or enzymatically. Older conventional methods of digesting lignocellulose used aqueous reaction systems and drastic reaction conditions such as high temperatures and high pressures using Brönsted acids. As a result of corrosion problems, large quantities of by-products and high plant costs, these processes have at present not been pursued further to a significant extent. As an alternative, the celloluse-comprising material can be subjected to a pretreatment in order to make the cellulose accessible to enzymatic hydrolysis. Thus, for example, the process of “steam explosion” uses high pressures in the presence or absence of acid catalysts in order to break up the microcrystalline structure of the cellulose and thus make efficient enzymatic hydrolysis possible. The corrosion problems can be countered by use of gaseous SO₂ or highly dilute aqueous sulfuric acid, but the process instead has other disadvantages. Thus, SO₂ is highly toxic and the large streams associated with the use of highly dilute H₂SO₄ lead to economic disadvantages. In addition, this form of pretreatment leads to long reaction times in the subsequent enzymatic hydrolysis with moderate enzyme activity and gives only moderate yield of glucose. None of the various known processes has therefore been implemented in the plants planned at present. Furthermore, there is a lack of a suitable process for the pretreatment of lignocellulose which makes rapid and very complete enzymatic degradation of the cellulose comprised therein possible. Even the dissolution of the complex composite structure of the biomass is problematical, since only few solvents for the strongly crosslinked biopolymers are known.

It is known that various ionic liquids can be used as solvents for cellulose. Thus, S. Zhu et al. in Green Chem. 2006, 8, pp. 325-327, describe in quite general terms the possibility of dissolve cellulose in ionic liquids and recovering it by addition of suitable precipitates such as water, ethanol, or acetone. As suitable ionic liquids, specific mention is made of 1-butyl-3-methylimidazolium chloride (BMIMCI) and 1-allyl-3-methylimidazolium chloride (AMIMCI).

EP-A-1 332 221 describes an enzyme catalysis in the presence of ionic liquids.

WO 03/029329 teaches dissolving cellulose in an ionic liquid which must comprise essentially no water and no nitrogen-comprising basis for further processing.

WO 2004/084627 describes a process for producing capsules of regenerated cellulose with an active substance in the interior, in which an ionic liquid is used as solvent.

DE 102005017733 describes solutions comprising cellulose, an ionic liquid as solvent and from 6 to 30% by weight of a nitrogen-comprising base, based on the total weight of the solution.

DE 10 2005 017 715 describes solutions comprising cellulose and an ionic liquid based on cations having at least one atom which is selected from among nitrogen, oxygen, sulfur and phosphorus and is present in protonated form.

The complex composite structure of lignocellulose, too, can be dissolved by ionic liquids. WO 2005/017001 describes a process for dissolving a lignocellulose material by means of an ionic liquid with irradiation with microwaves and/or under superatmospheric pressure and in the absence of water. The cations of the ionic liquid correspond to those mentioned in WO 2004/084627.

WO 2005/017252 describes a process for treating a lignocellulose material with an ionic liquid, e.g. for delignification.

In Green Chem. 2007, 9, pp. 63-69, D. A. Fort, R. C. Remsing, R. P. Swatloski, P. Moyna, G. Moyna and R. D. Rogers describe experiments on the dissolution of lignocellulose in the form of untreated wood in 1-butyl-3-methylimidazolium chloride and regeneration of the dissolved cellulose by precipitation using a precipitant.

In a poster presentation at the 28^{th} Symposium on Biotechnology for Fuels and Chemicals, Poster 2-61, Nashville, Tenn., USA, April 30-May 3, 2006, and in Biotechnology and Bioengineering, Vol. 95, No. 5, 2006, pp. 904-910 (published online on Aug. 17, 2006), A. P. Dadi, S. Varanasi and C. A. Schall describe the pretreatment of cellulose with 1-butyl-3-methylimidazolium chloride (BMIMCI) before enzyme-catalyzed hydrolysis to glucose. Here, the particular role of the chloride anion in the desired structural modification of the cellulose is emphasized. The small size of the anion, the high electronegativity and the high basicity are set to lead to particularly good attack on the free hydroxyl groups of the cellulose and thus to breaking-up of the crystalline structure. Nevertheless, this pretreatment process is still capable of improvement in a number of respects. Thus, the pretreatment of the cellulose is carried out under water-free conditions, which, inter alia, makes it necessary to work under a nitrogen atmosphere to avoid absorption of water. The extra complication associated with working in the absence of water is a significant disadvantage of this process. In addition, the chloride anion used is highly corrosive and is therefore unsuitable for use in an industrial process. The rate of enzymatic liberation of glucose, especially at the beginning of the reaction, is also in need of improvement.

In Chinese Science Bulletin 2006, Vol. 51, No. 20, pp. 2432-2436, L. Liying and C. Hongzhang describe the enzymatic hydrolysis of cellulose material which has been pretreated with 1-butyl-3-methylimidazolium chloride.

It has now surprisingly been found that ionic liquids based on polyatomic (multiatomic) anions are particularly advantageous for the pretreatment of lignocellulose materials for enzymatic hydrolysis to glucose.

The invention therefore provides a process for preparing a glucose product from a lignocellulose material, in which
a lignocellulose-comprising starting material is provided and treated with a liquid treatment medium which comprises an ionic liquid whose anions are selected from among polyatomic anions,
a cellulose-enriched material is isolated from the treated material and
the cellulose-enriched material is subjected to an enzymatic hydrolysis.

The process of the invention in its embodiments described below is advantageous in respect of one or more of the following points:
advantageous synthesis of the ionic liquids based on polyatomic anions which are used according to the invention;
simple and inexpensive pretreatment of the lignocellulose material;
more rapid enzymatic reaction of the cellulose-enriched material obtained from the pretreated lignocellulose material;
possibility of supplying the lignin comprised in the lignocellulose material to a separate use;
possibility of likewise subjecting the hemicellulose comprised in the lignocellulose material to an enzymatic hydrolysis to form sugars, e.g. arabinose and xylose;
avoidance of the formation of undesirable by-products, e.g. furfural or hydroxymethylfurfural, which act as inhibitors when the glucose is used in a subsequent fermentation;
possibility of reusing the ionic liquid employed;
possibility of forming closed product circuits for the digestion chemicals, precipitants and washing media used;
tolerance to water; the ionic liquids based on polyatomic anions which are used according to the invention generally tolerate the presence of water in an amount at which no precipitation of the cellulose from the treatment medium yet occurs;
no need to work under protective gas;
possibility of working at low temperatures;
lower amounts of enzyme based on substrate used;
possibility of higher substrate concentrations in the enzymatic hydrolysis;
the corrosion problems associated with the use of monoatomic anions, especially CI, do not occur; this is especially advantageous in reactor design.

It has surprisingly been found that the pretreatment of the lignocellulose material with an ionic liquid having polyatomic anions is of crucial importance for successful enzymatic degradation of the cellulose comprised in a lignocellulose material. Furthermore, it has surprisingly been found that the cellulose material used for the enzymatic hydrolysis can still comprise amounts of hemicellulose and/or lignin without the enzymatic hydrolysis being appreciably impaired.

The glucose product according to the invention can thus comprise not only glucose but also further sugars, e.g. from the enzymatic hydrolysis of hemicellulose, for example arabinose or xylose.

A fundamental advantage of the process of the invention is the opportunity to treat the cellulose-comprising starting material in the presence of water. The water content of the liquid treatment medium can be up to about 15% by weight. Naturally, the liquid treatment medium can also consist entirely of at least one ionic liquid.

For the purposes of the invention the term “solubilization” refers to conversion into a liquid state and comprises the production of solutions of the cellulose material and also conversion into a different solubilized state. If a cellulose material is converted into a solubilized state, the individual polymer molecules do not necessarily have to be completely surrounded by a solvation shell. The important thing is that the polymer goes into a liquid state as a result of the solubilization. Solubilizates within the meaning of the invention thus also include colloidal solutions, microdispersions, gels, etc. If undissolved material remains in the treatment of the lignocellulose-comprising starting material with the liquid treatment medium comprising the ionic liquid, this is not critical to the success of the process of the invention.

Lignocellulose forms the structural framework of the cell wall of a plant and comprises lignin, hemicelluloses and cellulose as main constituent. Further constituents are, for example, silicates, ash, extractable low molecular weight organic compounds (known as extractables, e.g. terpenes, resins, fats), polymers such as proteins, nucleic acids and plant gum (known as exudate), etc.

Lignin is a high molecular weight derivative of phenylpropane and has, depending on the source in nature, one or more methoxy groups on the phenyl rings and at least one hydroxy group on the propyl units. Hemicelluloses or polyoses are, like cellulose, made up of glycosidically linked sugar units (mainly arabinose and xylose), but the chains are more or less branched and the degree of polymerization is lower than in the case of cellulose (generally from about 50 to 250). Cellulose is a generally highly crystallized biopolymer of D-anhydroglucopyranose having long chains of sugar units linked by β-1,4-glycosidic bonds. The individual polymer chains are joined to one another by intermolecular and intramolecular hydrogen bonds and van der Waals interactions. The treatment according to the invention of the lignocellulose material with an ionic liquid leads to improved enzymatic hydrolysis of the resulting (regenerated) cellulose. It is assumed that the number of bonds in the polymer chain which are accessible to the enzyme is increased by the treatment. This is generally associated with a reduction in crystalline material and a corresponding increase in amorphous material, as can be determined, for example, by means of XRD.

The lignocellulose materials used according to the invention can be obtained, for example, from wood and plant fibers as starting material. These are preferably cellulose-rich natural fibers such as flax, hemp, sisal, jute, straw, coconut fibers, switchgrass (Panicum virgatum) and other natural fibers. Further suitable lignocellulose materials are the various types of wood, i.e. wood from broadleaved trees such as maple, beech, pear, oak, alder, ash, eucalyptus, hornbeam, cherry, lime, nut tree, poplar, willow, etc., and wood from conifers such as Douglas fir, spruce, yew, hemlock, pine, larch, fir, cedar, etc. Suitable lignocellulose materials are obtained, for example, as residues in the wood processing industry. They include not only scrap wood but also sawdust, parquetry grinding dust, etc. Suitable lignocellulose materials are also obtained as residues in agriculture, e.g. in the harvesting of cereals (wheat straw, maize straw, etc.), maize, sugar cane (bagasse), etc. Suitable lignocellulose materials are also obtained as residues in forestry, e.g. in the form of branches, bark, wood chips, etc. Another good source of lignocellulose materials are short rotation crops which make high biomass production on a relatively small area possible. A very good lignocellulose source is switchgrass.

The woody cell wall of central European timbers usually has approximately the following composition:

wood from broad leaved trees: cellulose 42-49%, hemicellulose 24-30%, lignin 25-30%, extractables 2-9%, ash (minerals) 0.2-0.8%;
wood from conifers: cellulose 42-51%, hemicellulose 27-40%, lignin 18-24%, extractables 1-10%, ash 0.2-0.8%.

For the purposes of the present patent application, ionic liquids are organic salts which are liquid at temperatures below 180° C. The ionic liquids preferably have a melting point of less than 150° C., particularly preferably less than 120° C., in particular less than 100° C. Ionic liquids which are present in the liquid state even at room temperature are described, for example, by K. N. Marsh et al., Fluid Phase Equilibria 219 (2004), 93-98 and J. G. Huddleston et al., Green Chemistry 2001, 3, 156-164.

Cations and anions are present in the ionic liquid. It is possible for a proton or an alkyl radical to be transferred from the cation to the anion within the ionic liquid, resulting in two uncharged molecules. Thus, an equilibrium between anions, cations and uncharged molecules formed therefrom can be present in the ionic liquid used according to the invention.

The ionic liquids used according to the invention have polyatomic, i.e. multiatomic, anions having two or more than two atoms.

For the purposes of the present invention, the expression “alkyl” comprises straight-chain or branched alkyl. Preference is given to straight-chain or branched C₁-C₃₀-alkyl, in particular C₁-C₁₈-alkyl and very particularly preferably C₁-C₁₂-alkyl. Examples of alkyl groups are, in particular, methyl, ethyl, n-propyl, isopropyl, n-butyl, isobutyl, sec-butyl, tert-butyl, n-pentyl, isopentyl, 1-methylbutyl, tert-pentyl, neopentyl, n-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, n-heptyl, n-octyl, 1-methylheptyl, 2-ethylhexyl, 2,4,4-trimethyl-pentyl, 1,1,3,3-tetramethylbutyl, n-nonyl, n-decyl, n-undecyl, n-dodecyl, n-tridecyl, n-tetradecyl, n-pentadecyl, n-hexadecyl, n-heptadecyl, n-octadecyl and n-eicosyl.

The expression alkyl also comprises alkyl radicals whose carbon chain can be interrupted by one or more nonadjacent heteroatoms or heteroatom-comprising groups which are preferably selected from among —O—, —S—, —NR^{a}—, —PR^{a}—, —SiR^{a}R^{aa} and/or —SO₂—. R^{a} is preferably hydrogen, alkyl, cycloalkyl, heterocycloalkyl, aryl or hetaryl. R^{aa} is preferably hydrogen, alkyl, cycloalkyl, heterocycloalkyl or aryl.

Examples of alkyl radicals whose carbon chains can be interrupted by one or two nonadjacent heteroatoms —O— are the following:

methoxymethyl, diethoxymethyl, 2-methoxyethyl, 2-ethoxyethyl, 2-propoxyethyl, diethoxyethyl, 2-butoxyethyl, 2-octyloxyethyl, 2-methoxypropyl, 3-methoxypropyl, 3-ethoxypropyl, 3-propoxypropyl, 2-isopropoxyethyl, 2-butoxypropyl, 3-butoxypropyl, 4-methoxybutyl, 4-ethoxybutyl, 4-propoxybutyl, 6-methoxyhexyl, 3,6-dioxaheptyl (5-methoxy-3-oxapentyl), 3,6-dioxaoctyl (7-methoxy-4-oxaheptyl), 4,8-dioxanonyl (7-methoxy-4-oxaheptyl), 3,7-dioxaoctyl, 3,7-dioxanonyl, 4,7-dioxaoctyl, 4,7-dioxanonyl, 2- and 4-butoxybutyl, 4,8-dioxadecyl, 9-ethoxy-5-oxanonyl.

Examples of alkyl radicals whose carbon chains can be interrupted by three or more nonadjacent heteroatoms —O— are oligooxyalkylenes and polyoxyalkylenes, i.e. compounds having repeating units which are preferably selected from among (CH₂CH₂O)ₓ₁, (CH(CH₃)CH₂O)ₓ₂ and ((CH₂)₄O)ₓ₃, where x1, x2 and x3 are each, independently of one another, an integer from 3 to 100, preferably from 3 to 80. The sum of x1, x2 and x3 is an integer from 3 to 300, in particular from 3 to 100. In polyoxyalkylenes having two or three different repeating units, any order is possible, i.e. the repeating units can be randomly distributed, alternate or be arranged in blocks. Examples are 3,6,9-trioxadecyl, 3,6,9-trioxaundecyl, 3,6,9-trioxadodecyl, 4,8,12-trioxamidecyl (11-methoxy-4,8-dioxaundecyl), 4,8,12-trioxatetradecyl, 14-methoxy-5,10-dioxatetradecyl, 5,10,15-trioxaheptadecyl, 3,6,9,12-tetraoxamidecyl, 3,6,9,12-tetraoxatetradecyl, 4,8,12,16-tetraoxaheptadecyl (15-methoxy-4,8,12-trioxapentadecyl), 4,8,12,16-tetraoxaoctadecyl and the like.

Examples of alkyl radicals whose carbon chains can be interrupted by one or more, e.g. 1, 2, 3, 4 or more than 4, nonadjacent heteroatoms —S— are the following:

butylthiomethyl, 2-methylthioethyl, 2-ethylthioethyl, 2-propylthioethyl, 2-butylthioethyl, 2-dodecylthioethyl, 3-methylthiopropyl, 3-ethylthiopropyl, 3-propylthiopropyl, 3-butylthiopropyl, 4-methylthiobutyl, 4-ethylthiobutyl, 4-propylthiobutyl, 3,6-dithiaheptyl, 3,6-dithiaoctyl, 4,8-dithianonyl, 3,7-dithiaoctyl, 3,7-dithianonyl, 2- and 4-butylthiobutyl, 4,8-dithiadecyl, 3,6,9-trithiadecyl, 3,6,9-trithiaundecyl, 3,6,9-trithiadodecyl, 3,6,9,12-tetrathiamidecyl and 3,6,9,12-tetrathiatetradecyl.

Examples of alkyl radicals whose carbon chains are interrupted by one or two nonadjacent heteroatom-comprising groups —NR^{a}— are the following:

2-monomethylaminoethyl and 2-monoethylaminoethyl, 2-dimethylaminoethyl, 3-methylaminopropyl, 2- and 3-dimethylaminopropyl, 3-monoisopropylaminopropyl, 2- and 4-monopropylaminobutyl, 2- and 4-dimethylaminobutyl, 6-methylaminohexyl, 6-dimethylaminohexyl, 6-methyl-3,6-diazaheptyl, 3,6-dimethyl-3,6-diazaheptyl, 3,6-diazaoctyl and 3,6-dimethyl-3,6-diazaoctyl.

Examples of alkyl radicals whose carbon chains can be interrupted by three or more nonadjacent heteroatom-comprising groups —NR^{a}— are oligoalkylenimines and polyalkylenimines. What has been said above with regard to the polyoxyalkylenes applies analogously to polyalkylenimines, with the oxygen atom being replaced in each case by an NR^{a} group, where R^{a} is preferably hydrogen or C₁-C₄-alkyl. Examples are 9-methyl-3,6,9-triazadecyl, 3,6,9-trimethyl-3,6,9-triazadecyl, 3,6,9-triazaundecyl, 3,6,9-trimethyl-3,6,9-triazaundecyl, 12-methyl-3,6,9,12-tetraazamidecyl, 3,6,9,12-tetramethyl-3,6,9,12-tetraazamidecyl and the like.

Examples of alkyl radicals whose carbon chains are interrupted by one or more, e.g. 1 or 2, nonadjacent —SO₂— groups are 2-methylsulfonylethyl, 2-ethylsulfonylethyl, 2-propylsulfonylethyl, 2-isopropylsulfonylethyl, 2-butylsulfonylethyl, 2-methylsulfonylpropyl, 3-methylsulfonylpropyl, 2-ethylsulfonylpropyl, 3-ethylsulfonylpropyl, 2-propylsulfonylpropyl, 3-propylsulfonylpropyl, 2-butylsulfonylpropyl, 3-butylsulfonylpropyl, 2-methylsulfonylbutyl, 4-methylsulfonylbutyl, 2-ethylsulfonylbutyl, 4-ethylsulfonylbutyl, 2-propylsulfonylbutyl, 4-propylsulfonylbutyl and 4-butylsulfonylbutyl.

The expression alkyl also comprises substituted alkyl radicals. Substituted alkyl groups can have, depending on the length of the alkyl chain, one or more (e.g. 1, 2, 3, 4, 5 or more than 5) substituents. These are preferably selected independently from among cycloalkyl, cycloalkyloxy, polycyclyl, polycyclyloxy, heterocycloalkyl, aryl, aryloxy, arylthio, hetaryl, halogen, hydroxy, SH, ═O, ═S, ═NR^{a}, COOH, carboxylate, SO₃H, sulfonate, NE¹E², nitro and cyano, where E¹ and E² are each, independently of one another, hydrogen, alkyl, cycloalkyl, heterocycloalkyl, aryl or hetaryl. Cycloalkyl, cycloalkyloxy, polycycloalkyl, polycycloalkyloxy, heterocycloalkyl, aryl and hetaryl substituents on the alkyl groups may in turn be unsubstituted or substituted; suitable substituents are those mentioned below for these groups.

What has been said above with regard to alkyl also applies in principle to the alkyl parts in alkoxy, alkylamino, dialkylamino, alkylthio(alkylsulfanyl), alkylsulfinyl, alkylsulfonyl, etc.

Suitable substituted alkyl radicals are the following:

alkyl which is substituted by carboxy, e.g. carboxymethyl, 2-carboxyethyl, 3-carboxypropyl, 4-carboxybutyl, 5-carboxypentyl, 6-carboxyhexyl, 7-carboxyheptyl, 8-carboxyoctyl, 9-carboxynonyl, 10-carboxydecyl, 12-carboxydodecyl and 14-carboxytetradecyl;
alkyl which is substituted by SO₃H, e.g. sulfomethyl, 2-sulfoethyl, 3-sulfopropyl, 4-sulfobutyl, 5-sulfopentyl, 6-sulfohexyl, 7-sulfoheptyl, 8-sulfooctyl, 9-sulfononyl, 10-sulfodecyl, 12-sulfododecyl and 14-sulfotetradecyl;
alkyl which is substituted by carboxylate, for example alkoxycarbonylalkyl, e.g. methoxycarbonylmethyl, ethoxycarbonylmethyl, n-butoxycarbonylmethyl, 2-methoxycarbonylethyl, 2-ethoxycarbonylethyl, 2-methoxycarbonylpropyl, 2-ethoxycarbonylpropyl, 2-(n-butoxycarbonyl)propyl, 2-(4-n-butoxycarbonyl)propyl, 3-methoxycarbonylpropyl, 3-ethoxycarbonylpropyl, 3-(n-butoxycarbonyl)propyl, 3-(4-n-butoxycarbonyl) propyl, aminocarbonylalkyl, e.g. aminocarbonylmethyl, aminocarbonylethyl, aminocarbonylpropyl and the like, alkylaminocarbonylalkyl such as methylaminocarbonylmethyl, methylaminocarbonylethyl, ethylcarbonylmethyl, ethylcarbonylethyl and the like, or dialkylaminocarbonylalkyl such as dimethylaminocarbonylmethyl, dimethylaminocarbonylethyl, dimethylcarbonylpropyl, diethylaminocarbonylmethyl, diethylaminocarbonylethyl, diethylcarbonylpropyl and the like.

Alkyl which is substituted by hydroxy, e.g. 2-hydroxyethyl, 2-hydroxypropyl, 3-hydroxypropyl, 3-hydroxybutyl, 4-hydroxybutyl, 2-hydroxy-2,2-dimethylethyl, 5-hydroxy-3-oxapentyl, 6-hydroxyhexyl, 7-hydroxy-4-oxaheptyl, 8-hydroxy-4-oxaoctyl, 8-hydroxy-3,6-dioxaoctyl, 9-hydroxy-5-oxanonyl, 11-hydroxy-4,8-dioxaundecyl, 11-hydroxy-3,6,9-trioxaundecyl, 14-hydroxy-5,10-dioxatetradecyl, 15-hydroxy-4,8,12-trioxapentadecyl and the like.

Alkyl which is substituted by amino, e.g. 2-aminoethyl, 2-aminopropyl, 3-aminopropyl, 4-aminobutyl, 6-aminohexyl and the like.

Alkyl which is substituted by cyano, e.g. 2-cyanoethyl, 3-cyanopropyl, 3-cyanobutyl and 4-cyanobutyl;
Alkyl which is substituted by halogen as defined below, with part or all of the hydrogen atoms in the alkyl group being able to be replaced by halogen atoms, e.g. C₁-C₁₈-fluoroalkyl, e.g. trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl and the like, C₁-C₁₈-chloroalkyl, e.g. chloromethyl, dichloromethyl, trichloromethyl, 2-chloroethyl, 2- and 3-chloropropyl, 2-, 3- and 4-chlorobutyl, 1,1-dimethyl-2-chloroethyl and the like, C₁-C₁₈-bromoalkyl, e.g. bromoethyl, 2-bromoethyl, 2- and 3-bromopropyl and 2-, 3- and 4-bromobutyl and the like.

Alkyl which is substituted by nitro, e.g. 2-nitroethyl, 2- and 3-nitropropyl and 2-, 3- and 4-nitrobutyl and the like.

Alkyl which is substituted by cycloalkyl, e.g. cyclopentylmethyl, 2-cyclopentylethyl, 3-cyclopentylpropyl, cyclohexylmethyl, 2-cyclohexylethyl, 3-cyclohexylpropyl and the like.

Alkyl which is substituted by ═O (oxo group), e.g. 2-oxopropyl, 2-oxobutyl, 3-oxobutyl, 1-methyl-2-oxopropyl, 2-oxopentyl, 3-oxopentyl, 1-methyl-2-oxobutyl, 1-methyl-3-oxobutyl, 2-oxohexyl, 3-oxohexyl, 4-oxohexyl, 2-oxoheptyl, 3-oxoheptyl, 4-oxoheptyl, 4-oxoheptyl and the like.

Alkyl which is substituted by ═S (thioxo group), e.g. 2-thioxopropyl, 2-thioxobutyl, 3-thioxobutyl, 1-methyl-2-thioxopropyl, 2-thioxopentyl, 3-thioxopentyl, 1-methyl-2-thioxobutyl, 1-methyl-3-thioxobutyl, 2-thioxohexyl, 3-thioxohexyl, 4-thioxohexyl, 2-thioxoheptyl, 3-thioxoheptyl, 4-thioxoheptyl, 4-thioxoheptyl and the like.

Alkyl which is substituted by ═NR^{a}—, preferably one in which R^{a} is hydrogen or C₁-C₄-alkyl, e.g. 2-iminopropyl, 2-iminobutyl, 3-iminobutyl, 1-methyl-2-iminopropyl, 2-iminopentyl, 3-iminopentyl, 1-methyl-2-iminobutyl, 1-methyl-3-iminobutyl, 2-iminohexyl, 3-iminohexyl, 4-iminohexyl, 2-iminoheptyl, 3-iminoheptyl, 4-iminoheptyl, 4-iminoheptyl, 2-methyliminopropyl, 2-methyliminobutyl, 3-methyliminobutyl, 1-methyl-2-methyliminopropyl, 2-methyliminopentyl, 3-methyliminopentyl, 1-methyl-2-methyliminobutyl, 1-methyl-3-methyliminobutyl, 2-methyliminohexyl, 3-methyliminohexyl, 4-methyliminohexyl, 2-methyliminoheptyl, 3-methyliminoheptyl, 4-methyliminoheptyl, 4-methyliminoheptyl, 2-ethyliminopropyl, 2-ethyliminobutyl, 3-ethyliminobutyl, 1-methyl-2-ethyliminopropyl, 2-ethyliminopentyl, 3-ethyliminopentyl, 1-methyl-2-ethyliminobutyl, 1-methyl-3-ethyliminobutyl, 2-ethyliminohexyl, 3-ethyliminohexyl, 4-ethyliminohexyl, 2-ethyliminoheptyl, 3-ethyliminoheptyl, 4-ethyliminoheptyl, 4-ethyliminoheptyl, 2-propyliminopropyl, 2-propyliminobutyl, 3-propyliminobutyl, 1-methyl-2-propyliminopropyl, 2-propyliminopentyl, 3-propyliminopentyl, 1-methyl-2-propyliminobutyl, 1-methyl-3-propyliminobutyl, 2-propyliminohexyl, 3-propyliminohexyl, 4-propyliminohexyl, 2-propyliminoheptyl, 3-propyliminoheptyl, 4-propyliminoheptyl, 4-propyliminoheptyl and the like.

Alkoxy is an alkyl group bound via an oxygen atom. Examples of alkoxy are: methoxy, ethoxy, n-propoxy, 1-methylethoxy, butoxy, 1-methylpropoxy, 2-methylpropoxy, 1,1-dimethylethoxy, n-pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy, hexoxy and R^{A}O—(CH₂CH₂CH₂CH₂O)ₙ—CH₂CH₂CH₂CH₂O— where R^{A} is hydrogen or C₁-C₄-alkyl, preferably hydrogen, methyl or ethyl, and n is from 0 to 10, preferably from 0 to 3.

Alkylthio(alkylsulfanyl) is an alkyl group bound via a sulfur atom. Examples of alkylthio are methylthio, ethylthio, propylthio, butylthio, pentylthio and hexylthio.

Alkylsulfinyl is an alkyl group bound via an S(═O) group.

Alkylsulfonyl is an alkyl group bound via an S(═O)₂ group.

Aryl radicals substituted by aryl (“arylalkyl”) have at least one unsubstituted or substituted aryl group as defined below. Suitable substituents on the aryl group are those mentioned below. Here, the alkyl group in “arylalkyl” can bear at least one further substituent as defined above and/or be interrupted by one or more nonadjacent heteroatoms or heteroatom-comprising groups selected from among —O—, —S—, —NR^{a}— and —SO₂—. Arylalkyl is preferably phenyl-C₁-C₁₀-alkyl, particularly preferably phenyl-C₁-C₄-alkyl, e.g. benzyl, 1-phenethyl, 2-phenethyl, 1-phenprop-1-yl, 2-phenprop-1-yl, 3-phenprop-1-yl, 1-phenbut-1-yl, 2-phenbut-1-yl, 3-phenbut-1-yl, 4-phenbut-1-yl, 1-phenbut-2-yl, 2-phenbut-2-yl, 3-phenbut-2-yl, 4-phenbut-2-yl, 1-(phenmeth)eth-1-yl, 1-(phenmethyl)-1-(methyl)eth-1-yl or -(phenmethyl)-1-(methyl)prop-1-yl; preferably benzyl and 2-phenethyl.

For the purposes of the present invention, the expression “alkenyl” comprises straight-chain and branched alkenyl groups which can, depending on the chain length, have one or more double bonds (e.g. 1, 2, 3, 4 or more than 4). Preference is given to C₂-C₁₈—, particularly preferably C₂-C₁₂-alkenyl groups. The expression “alkenyl” also comprises substituted alkenyl groups which can bear one or more (e.g. 1, 2, 3, 4, 5 or more than 5) substituents. Suitable substituents are selected, for example, from among ═O, ═S, ═NR^{a}, cycloalkyl, cycloalkyloxy, polycyclyl, polycyclyloxy, heterocycloalkyl, aryl, aryloxy, arylthio, hetaryl, halogen, hydroxy, SH, COOH, carboxylate, SO₃H, sulfonate, alkylsulfinyl, alkylsulfonyl, NE³E⁴, nitro and cyano, where E³ and E⁴ are each, independently of one another, hydrogen, alkyl, cycloalkyl, heterocycloalkyl, aryl or hetaryl.

The expression “alkenyl” also comprises alkenyl radicals whose carbon chain can be interrupted by one or more nonadjacent heteroatoms or heteroatom-comprising groups which are preferably selected from among —O—, —S—, —NR^{a}— and —SO₂—.

Alkenyl is then, for example, ethenyl(vinyl), 1-propenyl, 2-propenyl, 1-methylethenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like.

For the purposes of the present invention, the expression “cycloalkyl” comprises unsubstituted and substituted monocyclic saturated hydrocarbon groups which generally have from 3 to 12 ring carbons (C₃-C₁₂-cycloalkyl groups) such as cyclopropyl, cyclobutyl, cyclopentyl, cyclohexyl, cycloheptyl, cyclooctyl, cyclononyl, cyclodecyl, cycloundecyl or cyclododecyl, in particular C₅-C₁₂-cycloalkyl. Suitable substituents are generally selected from among alkyl, the substituents mentioned above for the alkyl groups, alkoxy and alkylthio. Suitable cycloalkyl groups can have one or more (e.g. 1, 2, 3, 4, 5 or more than 5) substituents, and in the case of halogen the cycloalkyl radical can be partially or fully substituted by halogen.

Examples of cycloalkyl groups are cyclopentyl, 2- and 3-methylcyclopentyl, 2- and 3-ethylcyclopentyl, chloropentyl, dichloropentyl, dimethylcyclopentyl, cyclohexyl, 2-, 3- and 4-methylcyclohexyl, 2-, 3- and 4-ethylcyclohexyl, 3- and 4-propylcyclohexyl, 3- and 4-isopropylcyclohexyl, 3- and 4-butylcyclohexyl, 3- and 4-sec-butylcyclohexyl, 3- and 4-tert-butylcyclohexyl, chlorohexyl, dimethylcyclohexyl, diethylcyclohexyl, methoxycyclohexyl, dimethoxycyclohexyl, diethoxycyclohexyl, butoxycyclohexyl, methylthiocyclohexyl, chlorocyclohexyl, dichlorocyclohexyl, cycloheptyl, 2-, 3- and 4-methylcycloheptyl, 2-, 3- and 4-ethylcycloheptyl, 3- and 4-propylcycloheptyl, 3- and 4-isopropylcycloheptyl, 3- and 4-butylcycloheptyl, 3- and 4-sec-butylcycloheptyl, 3- and 4-tert-butylcycloheptyl, cyclooctyl, 2-, 3-, 4- and 5-methylcyclooctyl, 2-, 3-, 4- and 5-ethylcyclooctyl, 3-, 4- and 5-propylcyclooctyl, partially fluorinated cycloalkyl and perfluorinated cycloalkyl of the formula CₙF_{2(n-a)-(1-b)}H_{2a-b} where n=5 to 12, 0<=a<=n and b=0 or 1.

Cycloalkyloxy is a cycloalkyl group as defined above bound via oxygen.

The expression “cycloalkenyl” comprises unsubstituted and substituted, monounsaturated or doubly unsaturated hydrocarbon groups having from 3 to 5, up to 8, up to 12 and preferably from 5 to 12, ring carbons, e.g. cyclopent-1-en-1-yl, cyclopent-2-en-1-yl, cyclopent-3-en-1-yl, cyclohex-1-en-1-yl, cyclohex-2-en-1-yl, cyclohex-3-en-1-yl, cyclohexa-2,5-dien-1-yl and the like. Suitable substituents are those mentioned above for cycloalkyl.

Cycloalkenyloxy is a cycloalkenyl group as defined above bound via oxygen.

For the purposes of the present invention, the expression “polycyclyl” comprises in the widest sense compounds which comprise at least two rings, regardless of how these rings are linked. The rings can be carbocyclic and/or heterocyclic. The rings can be saturated or unsaturated. The rings can be linked via a single or double bond (“multiring compounds”), be joined by fusion (“fused ring systems”) or be bridged (“bridged ring systems”, “cage compounds”). Preferred polycyclic compounds are bridged ring systems and fused ring systems. Fused ring systems can be aromatic, hydroaromatic and cyclic compounds linked by fusion (fused). Fused ring systems comprise two, three or more than three rings. Depending on the way in which the rings are linked, a distinction is made in the case of fused ring systems between ortho-fusion, i.e. each ring shares an edge or two atoms with each neighboring ring, and peri-fusion in which a carbon atom belongs to more than two rings. Among fused ring systems, preference is given to ortho-fused ring systems. For the purposes of the present invention, bridged ring systems include ones which do not belong to the multiring ring systems and not to the fused ring systems and in which at least two ring atoms belong to at least two different rings. Among bridged ring systems, a distinction is made according to the number of ring-opening reactions which are formally required to obtain an open-chain compound, between bicyclo, tricyclo, tetracyclo compounds, etc., which comprise two, three, four, etc., rings. The expression “bicycloalkyl” comprises bicyclic hydrocarbon radicals having preferably from 5 to 10 carbon atoms, e.g. bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl, bicyclo[4.4.0]decyl and the like. The expression “bicycloalkenyl” comprises monounsaturated bicyclic hydrocarbon radicals having preferably from 5 to 10 carbon atoms, e.g. bicyclo[2.2.1]hept-2-en-1-yl.

For the purposes of the present invention, the expression “aryl” comprises aromatic hydrocarbon radicals which may have one or more rings and be unsubstituted or substituted. Aryl is generally a hydrocarbon radical having from 6 to 10, up to 14, up to 18, preferably from 6 to 10, ring carbons. Aryl is preferably unsubstituted or substituted phenyl, naphthyl, anthracenyl, phenanthrenyl, naphthacenyl, chrysenyl, pyrenyl, etc., and particularly preferably phenyl or naphthyl. Substituted aryls can, depending on the number and size of their ring systems, have one or more (e.g. 1, 2, 3, 4, 5 or more than 5) substituents. These are preferably selected independently from among alkyl, alkoxy, cycloalkyl, cycloalkyloxy, heterocycloalkyl, aryl, aryloxy, arylthio, hetaryl, halogen, hydroxy, SH, alkylthio, alkylsulfinyl, alkylsulfonyl, COOH, carboxylate, SO₃H, sulfonate, NE⁵E⁶, nitro and cyano, where E⁵ and E⁶ are each, independently of one another, hydrogen, alkyl, cycloalkyl, cycloalkyloxy, polycyclylyl, polycyclyloxy, heterocycloalkyl, aryl, aryloxy or hetaryl. Aryl is particularly preferably phenyl, which if it is substituted can generally bear 1, 2, 3, 4 or 5, preferably 1, 2 or 3, substituents.

Aryl which bears one or more radicals is, for example, 2-, 3- and 4-methylphenyl, 2,4-, 2,5-, 3,5- and 2,6-dimethylphenyl, 2,4,6-trimethylphenyl, 2-, 3- and 4-ethylphenyl, 2,4-, 2,5-, 3,5- and 2,6-diethylphenyl, 2,4,6-triethylphenyl, 2-, 3- and 4-propylphenyl, 2,4-, 2,5-, 3,5- and 2,6-dipropylphenyl, 2,4,6-tripropylphenyl, 2-, 3- and 4-isopropylphenyl, 2,4-, 2,5-, 3,5- and 2,6-diisopropylphenyl, 2,4,6-triisopropylphenyl, 2-, 3- and 4-butylphenyl, 2,4-, 2,5-, 3,5- and 2,6-dibutylphenyl, 2,4,6-tributylphenyl, 2-, 3- and 4-isobutylphenyl, 2,4-, 2,5-, 3,5- and 2,6-diisobutylphenyl, 2,4,6-triisobutylphenyl, 2-, 3- and 4-sec-butylphenyl, 2,4-, 2,5-, 3,5- and 2,6-di-sec-butylphenyl, 2,4,6-tri-sec-butylphenyl, 2-, 3- and 4-tert-butylphenyl, 2,4-, 2,5-, 3,5- and 2,6-di-tert-butylphenyl, 2,4,6-tri-tert-butylphenyl and 2-, 3-, 4-dodecylphenyl; 2-, 3- and 4-methoxyphenyl, 2,4-, 2,5-, 3,5- and 2,6-dimethoxyphenyl, 2,4,6-trimethoxyphenyl, 2-, 3- and 4-ethoxyphenyl, 2,4-, 2,5-, 3,5- and 2,6-diethoxyphenyl, 2,4,6-triethoxyphenyl, 2-, 3- and 4-propoxyphenyl, 2,4-, 2,5-, 3,5- and 2,6-dipropoxyphenyl, 2-, 3- and 4-isopropoxyphenyl, 2,4-, 2,5-, 3,5- and 2,6-diisopropoxyphenyl, 2-, 3- and 4-butoxyphenyl, 2-, 3-, 4-hexyloxyphenyl; 2-, 3-, 4-chlorophenyl, 2,4-, 2,5-, 3,5- and 2,6-dichlorophenyl, trichlorophenyl, 2-, 3-, 4-fluorophenyl, 2,4-, 2,5-, 3,5- and 2,6-difluorophenyl, trifluorophenyl such as 2,4,6-trifluorophenyl, tetrafluorophenyl, pentafluorophenyl, 2-, 3- and 4-cyanophenyl; 2-nitrophenyl, 4-nitrophenyl, 2,4-dinitrophenyl, 2,6-dinitrophenyl; 4-dimethylaminophenyl; 4-acetylphenyl; methoxyethylphenyl, ethoxymethylphenyl; methylthiophenyl, isopropylthiophenyl or tert-butylthiophenyl; methylnaphthyl; isopropylnaphthyl or ethoxynaphthyl. Examples of substituted aryl in which two substituents which are bound to adjacent carbon atoms of the aryl ring form a fused ring or fused ring system are indenyl and fluorenyl.

For the purposes of the present invention, the expression “aryloxy” refers to an aryl bound via an oxygen atom.

For the purposes of the present invention, the expression “arylthio” refers to an aryl bound via a sulfur atom.

For the purposes of the present invention, the expression “heterocycloalkyl” comprises nonaromatic, unsaturated or fully saturated, cycloaliphatic groups which generally have from 5 to 8 ring atoms, preferably 5 or 6 ring atoms, and in which 1, 2 or 3 of the ring carbons have been replaced by heteroatoms selected from among oxygen, nitrogen, sulfur and an —NR^{a}— group and which are unsubstituted or substituted by one or more, for example, 1, 2, 3, 4, 5 or 6, C₁-C₆-alkyl groups. Examples of such heterocyclo-aliphatic groups are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothienyl, dihydrothienyl, tetrahydrofuranyl, dihydrofuranyl, tetrahydropyranyl, 1,2-oxazolin-5-yl, 1,3-oxazolin-2-yland dioxanyl. Nitrogen-comprising heterocycloalkyl can in principle be bound either via a carbon atom or via a nitrogen atom.

For the purposes of the present invention, the expression “heteroaryl(hetaryl)” comprises unsubstituted or substituted, heteroaromatic groups which have one or more rings and generally have from 5 to 14 ring atoms, preferably 5 or 6 ring atoms, and in which 1, 2 or 3 of the ring carbons have been replaced by one, two, three or four heteroatoms selected from among O, N, —NR^{a}— and S, e.g. furyl, thienyl, oxazolyl, isoxazolyl, thiazolyl, isothiazolyl, benzofuranyl, benzothiazolyl, benzimidazolyl, pyridyl, quinolinyl, acridinyl, pyridazinyl, pyrimidinyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl. If these heterocycloaromatic groups are substituted, they can generally bear 1, 2 or 3 substituents. The substituents are generally selected from among C₁-C₆-alkyl, C₁-C₆-alkoxy, hydroxy, carboxy, halogen and cyano.

5-to 7-membered nitrogen-comprising heterocycloalkyl or heteroaryl radicals which optionally comprise further heteroatoms are, for example, pyrrolyl, pyrazolyl, imidazolyl, triazolyl, pyrrolidinyl, pyrazolinyl, pyrazolidinyl, imidazolinyl, imidazolidinyl, pyridinyl, pyridazinyl, pyrimidinyl, pyrazinyl, triazinyl, piperidinyl, piperazinyl, oxazolyl, isooxazolyl, thiazolyl, isothiazolyl, indolyl, quinolinyl, isoquinolinyl or quinaldinyl, which may be unsubstituted or substituted as described above.

Halogen is fluorine, chlorine, bromine or iodine.

For the purposes of the present invention, carboxylate and sulfonate are preferably derivatives of a carboxylic acid function or a sulfonic acid function, in particular a metal carboxylate or sulfonate, a carboxylic ester or sulfonic ester function or a carboxamide or sulfonamide function. These include, for example, the esters with C₁-C₄-alkanoles such as methanol, ethanol, n-propanol, isopropanol, n-butanol, sec-butanol and tert-butanol.

For the purposes of the present invention, the expression “acyl” refers to alkanoyl, hetaroyl or aroyl groups having generally from 1 to 11, preferably from 2 to 8, carbon atoms, for example the formyl, acetyl, propanoyl, butanoyl, pentanoyl, hexanoyl, heptanoyl, 2-ethylhexanoyl, 2-propylheptanoyl, benzoyl or naphthoyl group.

The radicals E¹ and E², E³ and E⁴, E⁵ and E⁶ are selected independently from among hydrogen, alkyl, cycloalkyl and aryl. The groups NE¹E², NE³E⁴ and NE⁵E⁶ are preferably N,N-dimethylamino, N,N-diethylamino, N,N-dipropylamino, N,N-diisopropylamino, N,N-di-n-butylamino, N,N-di-tert-butylamino, N,N-dicyclohexylamino or N,N-diphenylamino.

In principle, all ionic liquids based on multiatomic anions are suitable for use in the process of the invention.

Preferred ionic liquids are
(A) salts of the general formula (I)

[A]ₙ⁺[Y]ⁿ⁻ (I),

where n is 1, 2, 3 or 4, [A]⁺ is a quaternary ammonium cation, an oxonium cation, a sulfonium cation or a phosphonium cation and [Y]ⁿ⁻ is a multiatomic, monovalent, divalent, trivalent or tetravalent anion or a mixture of these anions;
(B) mixed salts of the general formulae (II)

[A¹]⁺[A²]⁺[Y]ⁿ⁻ (II.a), where n=2,

[A¹]⁺[A²]⁺[A³]⁺[Y]ⁿ⁻ (II.b), where n=3,

[A¹]⁺[A²]⁺[A³]⁺[A⁴]⁺[Y]ⁿ⁻ (II.c), where n=4,

where [A¹]⁺, [A²]⁺, [A³]⁺ and [A⁴]⁺ are selected independently from among the groups mentioned for [A]⁺ and [Y]ⁿ⁻ is as defined under (A); or
(C) mixed salts of the general formulae (III)

[A¹]⁺[A²]⁺[A³]⁺[M¹]⁺[Y]ⁿ⁻ (III.a), where n=4,

[A¹]⁺[A²]⁺[M¹]⁺[M²]⁺[Y]ⁿ⁻ (III.b), where n=4,

[A¹]⁺[M¹]⁺[M²]⁺[M³]⁺[Y]ⁿ⁻ (III.c), where n=4,

[A¹]⁺[A²]⁺[M¹]⁺[Y]ⁿ⁻ (III.d), where n=3,

[A¹]⁺[M¹]⁺[M²]⁺[Y]ⁿ⁻ (III.e), where n=3,

[A¹]⁺[M¹]⁺[Y]ⁿ⁻ (III.f), where n=2,

[A¹]⁺[A²]⁺[M⁴]²⁺[Y]ⁿ⁻ (III.g), where n=4,

[A¹]⁺[M¹]⁺[M⁴]²⁺[Y]ⁿ⁻ (III.h), where n=4,

[A¹]⁺[M⁵]³⁺[Y]ⁿ⁻ (III.i), where n=4,

[A¹]⁺[M⁴]²⁺[Y]ⁿ⁻ (III.j), where n=3,

where [A¹]⁺, [A²]⁺ and [A³]⁺ are selected independently from among the groups mentioned for [A]⁺, [Y]ⁿ⁻ is as defined under (A) and [M¹]⁺, [M²]⁺, [M³]⁺ are monovalent metal cations, [M⁴]²⁺ is a divalent metal cation and [M⁵]³⁺ is a trivalent metal cation.

Preference is given to salts of groups A and B, particularly preferably group A.

The metal cations [M¹]⁺, [M²]⁺, [M³]⁺, [M⁴]²⁺ and [M⁵]³⁺ in the formulae (III.a) to (III.j) are generally metal cations of groups 1, 2, 6, 7, 8, 9, 10, 11, 12, 13 and 14 of the Periodic Table. Suitable metal cations are, for example, Li⁺, Na⁺, K⁺, Cs⁺, Mg²⁺, Ca²⁺, Ba²⁺, Cr³⁺, Fe²⁺, Fe³⁺, Co²⁺, Ni²⁺, Cu²⁺, Ag⁺, Zn²⁺ and Al³⁺.

Compounds which are suitable for forming the cation [A]⁺ of ionic liquids are described in DE 102 02 838 A1. These compounds preferably comprise at least one heteroatom, e.g. from 1 to to 10 heteroatoms, which is/are preferably selected from among nitrogen, oxygen, phosphorus and sulfur atoms. Preference is given to compounds which comprise at least one nitrogen atom and, if appropriate, additionally at least one further heteroatom which is different from nitrogen. Preference is given to compounds which comprise at least one nitrogen atom, particularly preferably from 1 to 10 nitrogen atoms, in particular from 1 to 5 nitrogen atoms, very particularly preferably from 1 to 3 nitrogen atoms and especially 1 or 2 nitrogen atoms. The latter nitrogen compounds can comprise further heteroatoms such as oxygen, sulfur or phosphorus atoms.

The nitrogen atom is, for example, a suitable carrier of the positive charge in the cation of the ionic liquid. If the nitrogen atom is the carrier of the positive charge in the cation of the ionic liquid, a cation can firstly be produced by quaternization of the nitrogen atom of, for instance, an amine or nitrogen heterocycle in the synthesis of the ionic liquids. Quaternization can be effected by protonation of the nitrogen atom. Depending on the protonation reagent used, salts having different anions are obtained. In cases in which it is not possible to form the desired anion in the quaternization itself, this can be brought about in a further step of the synthesis. Starting from, for example, an ammonium halide, the halide can be reacted with a Lewis acid to form a complex anion from the halide and Lewis acid. As an alternative, replacement of a halide ion by the desired anion is possible. This can be achieved by addition of a metal salt with precipitation of the metal halide formed, by means of an ion exchanger or by displacement of the halide ion by a strong acid (with liberation of the hydrogen halide). Suitable methods are described, for example, in Angew. Chem. 2000, 112, pp. 3926-3945, and the references cited therein.

Preference is given to compounds which comprise at least one five- or six-membered heterocycle, in particular a five-membered heterocycle, which has at least one nitrogen atom and, if appropriate, an oxygen or sulfur atom. Particular preference is given to compounds which comprise at least one five- or six-membered heterocycle having one, two or three nitrogen atoms and a sulfur or oxygen atom, very particularly preferably compounds having two nitrogen atoms. Further preference is given to aromatic heterocycles.

Particularly preferred compounds are compounds which have a molar mass of less than 1000 g/mol, very particularly preferably less than 800 g/mol and in particular less than 500 g/mol.

Preferred cations are selected from the compounds of the formulae (IV.a) to (IV.w),

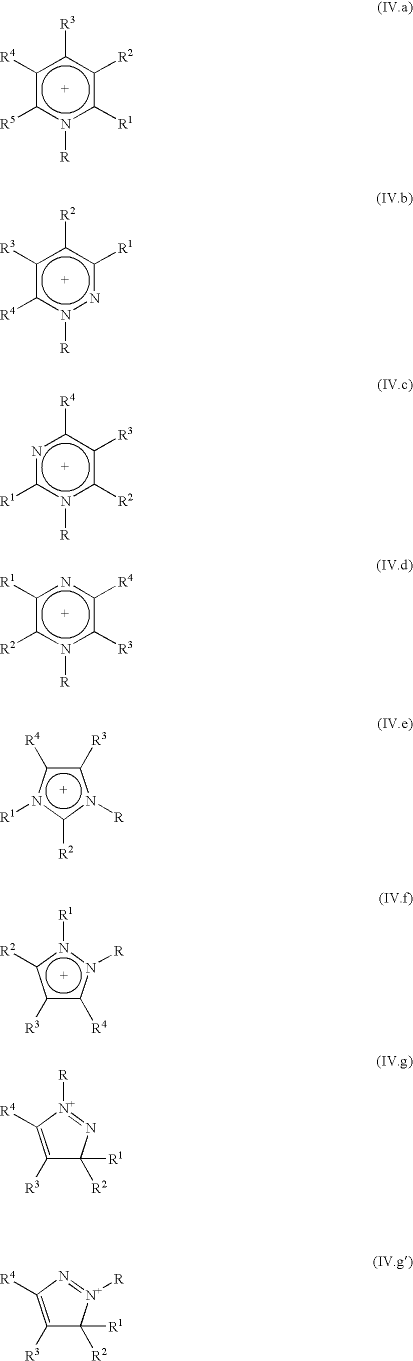

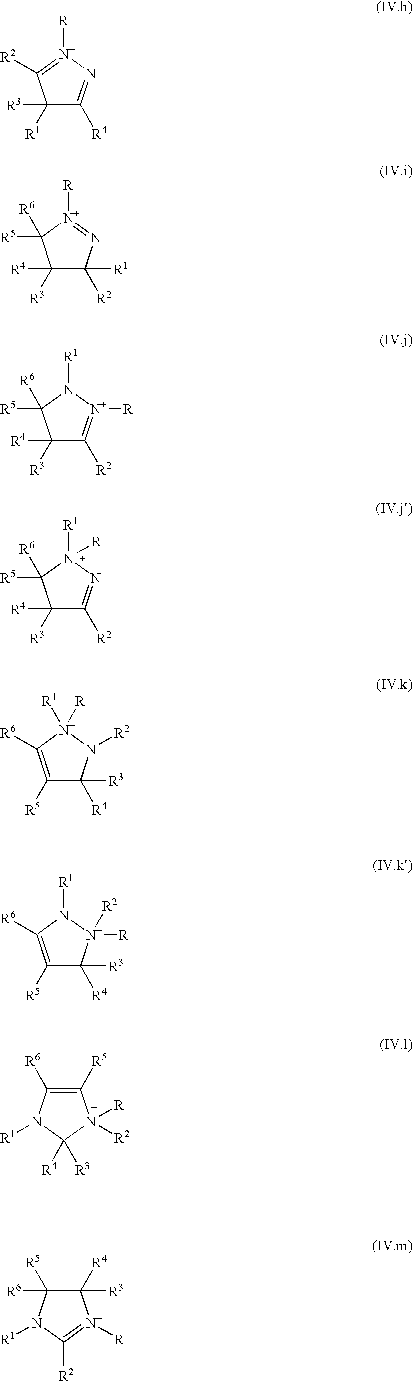

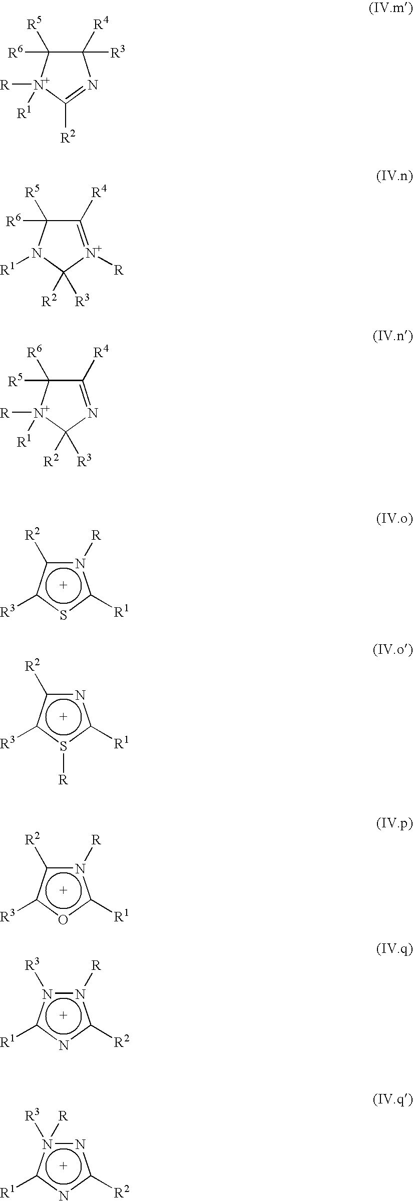

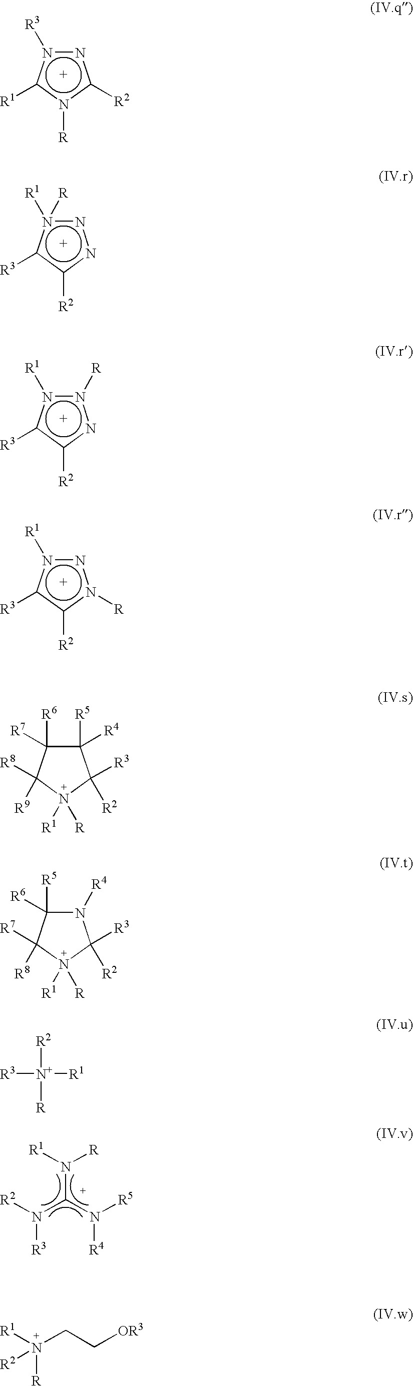

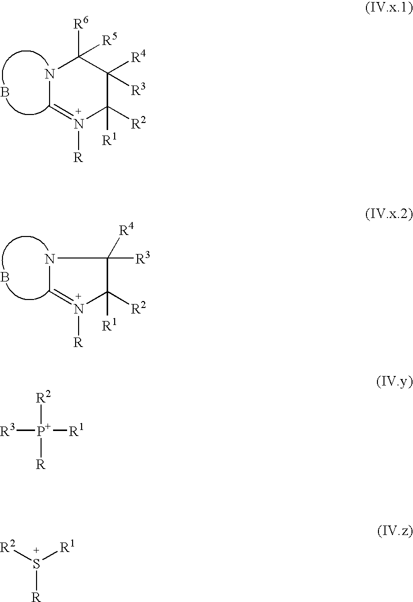

and oligomers comprising these structures, where
R is hydrogen, alkyl, alkenyl, cycloalkyl, cycloalkenyl, polycyclyl, heterocycloalkyl, aryl or heteroaryl;
radicals R¹, R², R³, R⁴, R⁵, R⁶, R⁷, R⁸ and R⁹ which are bound to a ring carbon are each, independently of one another, hydrogen, a sulfo group, COOH, carboxylate, sulfonate, acyl, alkoxycarbonyl, cyano, halogen, hydroxyl, SH, nitro, NE¹E², alkyl, alkoxy, alkylthio, alkylsulfinyl, alkylsulfonyl, alkenyl, cycloalkyl, cycloalkyloxy, cycloalkenyl, cycloalkenyloxy, polycyclyl, polycyclyloxy, heterocycloalkyl, aryl, aryloxy or heteroaryl, where E¹ and E² are each, independently of one another, hydrogen, alkyl, cycloalkyl, heterocycloalkyl, aryl or hetaryl,
radicals R¹, R², R³, R⁴, R⁵, R⁶, R⁷, R⁸ and R⁹ which are bound to a ring heteroatom are each hydrogen, SO₃H, NE¹E², alkyl, alkoxy, alkenyl, cycloalkyl, cycloalkenyl, polycyclyl, heterocycloalkyl, aryl or heteroaryl, where E¹ and E² are each, independently of one another, hydrogen, alkyl, cycloalkyl, heterocycloalkyl, aryl or hetaryl, or
   two adjacent radicals R¹ to R⁹ together with the ring atoms to which they are bound may also form at least one fused, saturated, unsaturated or aromatic ring or ring system which has from 1 to 30 carbon atoms and may comprise from 1 to 5 nonadjacent heteroatoms or heteroatom-comprising groups and be unsubstituted or substituted, and
   two geminal radicals R¹ to R⁹ may also together be ═O, ═S or ═NR^{b}, where R^{b} is hydrogen, alkyl, cycloalkyl, aryl or heteroaryl, and
   R¹ and R³ or R³ and R⁵ in the compounds of the formula (IV.x.1) may together also be the second part of a double bond between the ring atoms bearing these radicals, and
B in the compounds of the formulae (IV.x.1) and (IV.x.2) together with the C—N group to which it is bound forms a 4- to 8-membered, saturated or unsaturated or aromatic ring which may optionally be substituted and/or may optionally have further heteroatoms or heteroatom-comprising groups and/or may comprise further fused saturated, unsaturated or aromatic carbocycles or heterocycles.

As regards the general meanings of the abovementioned radicals carboxylate, sulfonate, acyl, alkoxycarbonyl, halogen, NE¹E², alkyl, alkoxy, alkylthio, alkylsulfinyl, alkylsulfonyl, alkenyl, cycloalkyl, cycloalkyloxy, cycloalkenyl, cycloalkenyloxy, polycyclyl, polycyclyloxy, heterocycloalkyl, aryl, aryloxy or heteroaryl, what has been said above is hereby fully incorporated by reference. Radicals R¹ to R⁹ which are bound to a carbon atom in the abovementioned formulae (IV) and have a heteroatom or a heteroatom-comprising group can also be bound directly via a heteroatom to the carbon atom.

If two adjacent radicals R¹ to R⁹ together with the ring atoms to which they are bound form at least one fused, saturated, unsaturated or aromatic ring or ring system which has from 1 to 30 carbon atoms and may have from 1 to 5 nonadjacent heteroatoms or heteroatom-comprising groups and be unsubstituted or substituted, these radicals can together preferably form, as fused-on building blocks, 1,3-propylene, 1,4-butylene, 1,5-pentylene, 2-oxa-1,3-propylene, 1-oxa-1,3-propylene, 2-oxa-1,3-propylene, 1-oxa-1,3-propenylene, 3-oxa-1,5-pentylene, 1-aza-1,3-propenylene, 1-C₁-C₄-alkyl-1-aza-1,3-propenylene, 1,4-buta-1,3-dienylene, 1-aza-1,4-buta-1,3-dienylene or 2-aza-1,4-buta-1,3-dienylene.

The radical R is preferably
unsubstituted C₁-C₁₈-alkyl such as methyl, ethyl, 1-propyl, 2-propyl, 1-butyl, 2-butyl, 2-methyl-1-propyl(isobutyl), 2-methyl-2-propyl(tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl and 1-octadecyl;
C₁-C₁₈-alkyl which is substituted by one or more hydroxyl, halogen, phenyl, cyano, C₁-C₆-alkoxycarbonyl and/or SO₃H groups, especially hydroxy-C₁-C₁₈-alkyl such as 2-hydroxyethyl or 6-hydroxyhexyl; phenyl-C₁-C₁₈-alkyl such as benzyl, 3-phenylpropyl; cyano-C₁-C₁₈-alkyl such as 2-cyanoethyl; C₁-C₆-alkoxy-C₁-C₁₈-alkyl such as 2-(methoxycarbonyl)ethyl, 2-(ethoxycarbonyl)ethyl or 2-(n-butoxycarbonyl)ethyl; C₁-C₁₈-fluoroalkyl such as trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl; sulfo-C₁-C₁₈-alkyl such as 3-sulfopropyl;
hydroxyethyloxyalkyl, radicals of oligoalkylene and polyalkylene glycols such as polyethylene glycols and polypropylene glycols and their oligomers having from 2 to 100 units and a hydrogen or a C₁-C₈-alkyl as end group, for example R^{A}O—(CHR^{B}—CH₂—O)ₙ—CHR^{B}—CH₂— where R^{A} and R^{B} are preferably each hydrogen, methyl or ethyl and n is preferably from 0 to 3, in particular 3-oxabutyl, 3-oxapentyl, 3,6-dioxaheptyl, 3,6-dioxaoctyl, 3,6,9-trioxadecyl, 3,6,9-trioxaundecyl, 3,6,9,12-tetraoxamidecyl and 3,6,9,12-tetraoxatetradecyl; and
C₂-C₆-alkenyl such as vinyl or propenyl.

The radical R is particularly preferably linear C₁-C₁₈-alkyl such as methyl, ethyl, 1-propyl, 1-butyl, 1-pentyl, 1-hexyl, 1-heptyl, 1-octyl, 1-decyl, 1-dodecyl, 1-tetradecyl, 1-hexadecyl, 1-octadecyl, very particularly preferably methyl, ethyl, 1-butyl or 1-octyl, or CH₃O—(CH₂CH₂O)ₙ—CH₂CH₂— and CH₃CH₂O—(CH₂CH₂O)ₘ—CH₂CH₂— where m is from 0 to 3.

Preference is given to the radicals R¹ to R⁹ each being, independently of one another,
hydrogen;
halogen;
a functional group selected from among hydroxy, alkoxy, alkylthio, carboxyl, —COOH, sulfonate, cyano, acyl, alkoxycarbonyl, NE¹E² and nitro, where E¹ and E² are as defined above;
C₁-C₁₈-alkyl which is unsubstituted or substituted as defined above and/or may be interrupted as defined above by at least one heteroatom or a heteroatom-comprising group;
C₂-C₁₈-alkenyl which is unsubstituted or substituted as defined above or may be interrupted as defined above by at least one heteroatom;
C₆-C₁₀-aryl which is unsubstituted or substituted as defined above;
C₅-C₁₂-cycloalkyl which is unsubstituted or substituted as defined above;
polycyclyl which is unsubstituted or substituted as defined above;
C₅-C₁₂-cycloalkenyl which is unsubstituted or substituted as defined above;
heterocycloalkyl which has 5 or 6 ring atoms and in which the ring has 1, 2 or 3 heteroatoms or heteroatom-comprising groups selected from among oxygen, nitrogen, sulfur and NR^{a} in addition to ring carbons and which is unsubstituted or substituted as defined above;
heteroaryl which has from 5 to 10 ring atoms and in which the ring has 1, 2 or 3 heteroatoms or heteroatom-comprising groups selected from among oxygen, nitrogen, sulfur and NR^{a} in addition to ring carbons and which is unsubstituted or substituted as defined above.

Preference is likewise given to two adjacent radicals R¹ to R⁹ together with the ring atoms to which they are bound forming a fused, saturated, unsaturated or aromatic ring or ring system which has from 1 to 12 carbon atoms and can have from 1 to 5 nonadjacent heteroatoms or heteroatom-comprising groups which are preferably selected from among oxygen, nitrogen, sulfur and NR^{a} and is unsubstituted or may be substituted by substituents which are preferably selected independently from among alkoxy, cycloalkyl, cycloalkoxy, polycyclyl, polycyclyloxy, heterocycloalkyl, aryl, aryloxy, arylthio, heteroaryl, halogen, hydroxy, SH, ═O, ═S, ═NR^{a}, COOH, carboxylate, —SO₃H, sulfonate, NE¹E², nitro and cyano, where E¹ and E² are each, independently of one another, hydrogen, alkyl, cycloalkyl, heterocycloalkyl, aryl or hetaryl.

When R¹ to R⁹ are alkoxy, then R¹ to R⁹ are preferably methoxy or ethoxy or R^{A}O—(CH₂CH₂CH₂CH₂O)ₙ—CH₂CH₂CH₂CH₂O— where R^{A} and R^{B} are preferably each hydrogen, methyl or ethyl and n is preferably from 0 to 3.

When R¹ to R⁹ are acyl, then R¹ to R⁹ are preferably formyl or C₁-C₄-alkylcarbonyl, in particular formyl or acetyl.

When R¹ to R⁹ are C₁-C₁₈-alkyl, then R¹ to R⁹ are preferably unsubstituted C₁-C₁₈-alkyl such as methyl, ethyl, 1-propyl, 2-propyl, 1-butyl, 2-butyl, 2-methyl-1-propyl(isobutyl), 2-methyl-2-propyl(tert-butyl), 1-pentyl, 2-pentyl, 3-pentyl, 2-methyl-9-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, 2-ethylhexyl, 2,4,4-trimethylpentyl, 1,1,3,3-tetramethylbutyl, 1-nonyl, 1-decyl, 1-undecyl, 1-dodecyl, 1-tridecyl, 1-tetradecyl, 1-pentadecyl, 1-hexadecyl, 1-heptadecyl, 1-octadecyl;

C₁-C₁₈-halogenalkyl, especially C₁-C₁₈-fluoroalkyl, for example trifluoromethyl, difluoromethyl, fluoromethyl, pentafluoroethyl, heptafluoropropyl, heptafluoroisopropyl, nonafluorobutyl, nonofluoroisobutyl, undecylfluoropentyl, undecylisopentyl, C6F13, C₈F₁₇, C₁₀F₂₁, C₁₂F₂₅, especially C₁-C₁₈-chloroalkyl such as chloromethyl, 2-chloroethyl, trichloromethyl, 1,1-dimethyl-2-chloroethyl;
amino-C₁-C₁₅-alkyl, such as 2-aminoethyl, 2-aminopropyl, 3-aminopropyl, 4-aminobutyl, 6-aminohexyl,
C₁-C₆-alkylamino-C₁-C₁₈-alkyl such as 2-methylaminoethyl, 2-methylaminopropyl, 3-methylaminopropyl, 4-methylaminobutyl, 6-methylaminohexyl;
di(C₁-C₆-alkyl)-C₁-C₁₈-alkyl such as 2-dimethylaminoethyl, 2-dimethylaminopropyl, 3-di methylaminopropyl, 4-dimethylaminobutyl, 6-dimethylaminohexyl, cyano-C₁-C₁₈-alkyl such as 2-cyanoethyl, 2-cyanopropyl, C₁-C₁₀-alkoxy-C₁-C₁₈-alkyl such as methoxymethyl, 2-methoxyethyl, 2-methoxypropyl, 3-methoxypropyl, 2-methoxyisopropyl, 4-methoxybutyl, 6-methoxyhexyl, 2-ethoxyethyl, 2-ethoxypropyl, 3-ethoxypropyl, 4-ethoxybutyl, 6-ethoxyhexyl, 2-isopropoxyethyl, 2-butoxyethyl, 2-butoxypropyl, 2-octyloxyethyl, 5-methoxy-3-oxapentyl, 8-methoxy-3,6-dioxaoctyl, 7-methoxy-4-oxaheptyl, 11-methoxy-4,8-dioxaundecyl, 9-methoxy-5-oxanonyl, 9-methoxy-5-oxanonyl, 14-methoxy-5,10-dioxatetradecyl, 5-ethoxy-3-oxapentyl, 8-ethoxy-3,6-dioxaoctyl, 7-ethoxy-4-oxaheptyl, 11-ethoxy-4,8-dioxaundecyl, 9-ethoxy-5-oxanonyl or 14-ethoxy-5,10-oxatetradecyl, 15-methoxy-4,8,12-trioxapentadecyl, 11-methoxy-3,6,9-trioxaundecyl, 11-ethoxy-3,6,9-trioxaundecyl, 15-ethoxy-4,8,12-trioxapentadecyl;
di(C₁-C₁₀-alkoxy-C₁-C₁₈-alkyl) such as diethoxymethyl or diethoxyethyl, C₁-C₆-alkoxycarbonyl-C₁-C₁₈-alkyl such as 2-(methoxycarbonyl)ethyl, 2-(ethoxycarbonyl)ethyl, 2-(n-butoxycarbonyl)ethyl;
di(C₁-C₆-alkoxycarbonyl)-C₁-C₁₈-alkyl such as 1,2-di(methoxycarbonyl)ethyl, hydroxy-C₁-C₁₈-alkyl such as 2-hydroxyethyl, 2-hydroxypropyl, 3-hydroxypropyl, 4-hydroxybutyl, 6-hydroxyhexyl, 2-hydroxy-2,2-dimethylethyl, 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapentadecyl, 9-hydroxy-5-oxanonyl, 14-hydroxy-5,10-dioxatetradecyl;
C₁-C₁₂-alkylsulfanyl-C₁-C₁₈-alkyl such as butylthiomethyl, 2-dodecylthioethyl, C₅-C₁₂-cycloalkyl-C₁-C₁₈-alkyl such as cyclopentylmethyl, 2-cyclopentylethyl, 3-cyclopentylpropyl, cyclohexylmethyl, 2-cyclohexylethyl, 3-cyclohexylpropyl, phenyl-C₁-C₁₈-alkyl, where the phenyl part of phenyl-C₁-C₁₅-alkyl is unsubstituted or substituted by one, two, three or four substituents selected independently from among C₁-C₁₈-alkyl, halogen, C₁-C₆-alkoxy and nitro, e.g. benzyl(phenylmethyl), 1-phenylethyl, 2-phenylethyl, 3-phenylpropyl, p-tolylmethyl, 1-(p-butylphenyl)ethyl, p-chlorobenzyl, 2,4-dichlorobenzyl, p-methoxybenzyl, m-ethoxybenzyl, phenyl-C(CH₃)₂—, 2,6-dimethylphenylmethyl,
diphenyl-C₁-C₁₈-alkyl such as diphenylmethyl(benzhydryl);
triphenyl-C₁-C₁₈-alkyl such as triphenylmethyl;
phenoxy-C₁-C₁₈-alkyl such as 2-phenoxyethyl, 2-phenoxypropyl, 3-phenoxypropyl, 4-phenoxybutyl, 6-phenoxyhexyl;
phenylthio-C₁-C₁₈-alkyl such as 2-phenylthioethyl.

When R¹ to R⁹ are C₂-C₁₈-alkenyl, then R¹ to R⁹ are preferably C₂-C₈-alkenyl such as vinyl, 2-propenyl, 3-butenyl, cis-2-butenyl, trans-2-butenyl or C₂-C₁₈-alkenyl which is partially or fully substituted by fluorine.

When R¹ to R⁹ are C₆-C₁₀-aryl, then R¹ to R⁹ are preferably phenyl or naphthyl, where phenyl or naphthyl is unsubstituted or substituted by one, two, three or four substituents selected independently from among halogen, C₁-C₁₅-alkyl, C₁-C₆-alkoxy, C₁-C₆-alkylsulfanyl, C₁-C₆-alkoxy-C₁-C₆-alkyl, C₁-C₆-alkylcarbonyl, amino, C₁-C₆-alkylamino, di(C₁-C₆-dialkyl)amino and nitro, e.g. phenyl, methylphenyl(tolyl), dimethylphenyl(xylyl) such as 2,6-dimethylphenyl, trimethylphenyl such as 2,4,6-trimethylphenyl, ethylphenyl, diethylphenyl, isopropylphenyl, tert-butylphenyl, dodecylphenyl, chlorophenyl, dichlorophenyl, trichlorophenyl, fluorophenyl, difluorophenyl, trifluorophenyl, tetrafluorophenyl, pentafluorophenyl, 2,6-dichlorophenyl, 4-bromophenyl, methoxyphenyl, dimethoxyphenyl, ethoxyphenyl, hexyloxyphenyl, 2,6-dimethoxyphenyl, 2-nitrophenyl, 4-nitrophenyl, 2,4-dinitrophenyl, 2,6-dinitrophenyl, 4-dimethylaminophenyl, 4-acetylphenyl, methoxyethylphenyl, ethoxymethylphenyl, methylthiophenyl, isopropylthiophenyl, tert-butylthiophenyl, α-naphthyl, β-naphthyl, methylnaphthyl, isopropylnaphthyl, chloronaphthyl, ethoxynaphthyl or partially fluorinated phenyl or perfluorinated phenyl.

When R¹ to R⁹ are C₅-C₁₂-cycloalkyl, then R¹ to R⁹ are preferably unsubstituted cycloalkyl such as cyclopentyl or cyclohexyl;

C₅-C₁₂-cycloalkyl substituted by one or two substituents selected independently from among C₁-C₆-alkyl, C₁-C₆-alkoxy, C₁-C₆-alkylsulfanyl and chlorine, e.g. butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl;
C₅-C₁₂-cycloalkyl which is completely or fully fluorinated.

When R¹ to R⁹ are polycyclyl, then R¹ to R⁹ are preferably C₅-C₁₂-bicycloalkyl such as norbornyl or C₅-C₁₂-bicycloalkenyl such as norbornenyl.

When R¹ to R⁹ are C₅-C₁₂-cycloalkenyl, then R¹ to R⁹ are preferably unsubstituted cycloalkenyl such as cyclopent-2-en-1-yl, cyclopent-3-en-1-yl, cyclohex-2-en-1-yl, cyclohex-1-en-1-yl, cyclohexa-2,5-dien-1-yl or partially or fully fluorinated cycloalkenyl.

When R¹ to R⁹ are heterocycloalkyl having 5 or 6 ring atoms, then R¹ to R⁹ are preferably 1,3-dioxolan-2-yl, 1,3-dioxan-2-yl, 2-methyl-1,3-dioxolan-2-yl, 4-methyl-1,3-dioxolan-2-yl.

When R¹ to R⁹ are heteroaryl, then R¹ to R⁹ are preferably furyl, thienyl, pyrryl, pyridyl, indolyl, benzoxazolyl, benzimidazolyl, benzothiazolyl. If the hetaryl group is substituted, it bears 1, 2 or 3 substituents selected independently from among C₁-C₆-alkyl, C₁-C₆-alkoxy and halogen, for example dimethylpyridyl, methylquinolyl, dimethylpyrryl, methoxyfuryl, dimethoxypyridyl or difluoropyridyl.

Particular preference is given to the radicals R¹ to R⁹ each being, independently of one another,
hydrogen;
unbranched or branched C₁-C₁₈-alkyl which is unsubstituted or substituted by one or more hydroxy, halogen, phenyl, cyano, C₁-C₆-alkoxycarbonyl and/or sulfo groups, for example methyl, ethyl, 1-propyl, 2-propyl, 1-butyl, 2-butyl, 2-methyl-1-propyl(isobutyl), 2-methyl-2-propyl(tert-butyl), 1-pentyl, 2-pentyl, 3-pentyl, 2-methyl-1-butyl, 3-methyl-1-butyl, 2-methyl-2-butyl, 3-methyl-2-butyl, 2,2-di-methyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexa-decyl, 1-octadecyl, 2-hydroxyethyl, benzyl, 3-phenylpropyl, 2-cyanoethyl, methoxycarbonylmethyl, ethoxycarbonylmethyl, n-butoxycarbonylmethyl, tert-butoxycarbonylmethyl, 2-(methoxycarbonyl)ethyl, 2-(ethoxycarbonyl)ethyl, 2-(n-butoxycarbonyl)ethyl, trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl, 6-hydroxyhexyl and 3-sulfopropyl;
hydroxyethyloxyalkyl, radicals of oligoalkylene and polyalkylene glycols such as polyethylene glycols and polypropylene glycols and their oligomers having from 2 to 100 units and a hydrogen or a C₁-C₈-alkyl as end group, for example R^{A}O—(CHR^{B}—CH₂—O)ₙ—CHR^{B}—CH₂— or R^{A}O—(CH₂CH₂CH₂CH₂O)ₙ—CH₂CH₂CH₂CH₂O— where R^{A} and R^{B} are preferably each hydrogen, methyl or ethyl and n is preferably from 0 to 3, in particular 3-oxabutyl, 3-oxapentyl, 3,6-dioxaheptyl, 3,6-dioxaoctyl, 3,6,9-trioxadecyl, 3,6,9-trioxaundecyl, 3,6,9,12-tetraoxamidecyl and 3,6,9,12-tetraoxatetradecyl;
C₂-C₄-alkenyl such as vinyl and allyl; and
N,N-di-C₁-C₆-alkylamino such as N,N-dimethylamino and N,N-diethylamino.

Very particular preference is given to the radicals R¹ to R⁹ each being, independently of one another, hydrogen; C₁-C₁₈-alkyl such as methyl, ethyl, 1-butyl, 1-pentyl, 1-hexyl, 1-heptyl, 1-octyl; phenyl; 2-hydroxyethyl; 2-cyanoethyl; 2-(alkoxycarbonyl)ethyl such as 2-(methoxycarbonyl)ethyl, 2-(ethoxycarbonyl)ethyl or 2-(n-butoxycarbonyl)ethyl; N,N—(C₁-C₄-dialkyl)amino such as N,N-dimethylamino or N,N-diethylamino; chlorine and radicals of oligoalkylene glycol, e.g. CH₃O—(CH₂CH₂O)ₙ—CH₂CH₂— or CH₃CH₂O—(CH₂CH₂O)ₙ—CH₂CH₂— where n is from 0 to 3.

Very particularly preferred pyridinium ions (IVa) are those in which
one of the radicals R¹ to R⁵ is methyl, ethyl or chlorine and the remaining radicals R¹ to R⁵ are each hydrogen;

R³ is dimethylamino and the remaining radicals R¹, R², R⁴ and R⁵ are each hydrogen;
all radicals R¹ to R⁵ are hydrogen;

R² is carboxy or carboxamide and the remaining radicals R¹, R², R⁴ and R⁵ are each hydrogen; or

R¹ and R² or R² and R³ are together 1,4-buta-1,3-dienylene and the remaining radicals R¹, R³, R⁴ and R⁵ are each hydrogen;

and in particular those in which
R¹ to R⁵ are each hydrogen; or
one of the radicals R¹ to R⁵ is methyl or ethyl and the remaining radicals R¹ to R⁵ are each hydrogen.

As particularly preferred pyridinium ions (IVa), mention may be made of pyridinium, 2-methylpyridinium, 2-ethylpyridinium, 5-ethyl-2-methylpyridinium and 2-methyl-3-ethylpyridinium and also 1-methylpyridinium, 1-ethylpyridinium, 1-(1-butyl)pyridinium, 1-(1-hexyl)pyridinium, 1-(1-octyl)pyridinium, 1-(1-hexyl)pyridinium, 1-(1-octyl)pyridinium, 1-(1-dodecyl)pyridinium, 1-(1-tetradecyl)pyridinium, 1-(1-hexadecyl)-pyridinium, 1,2-dimethylpyridinium, 1-ethyl-2-methylpyridinium, 1-(1-butyl)-2-methylpyridinium, 1-(1-hexyl)-2-methylpyridinium, 1-(1-octyl)-2-methylpyridinium, 1-(1-dodecyl)-2-methylpyridinium, 1-(1-tetradecyl)-2-methylpyridinium, 1-(1-hexadecyl)-2-methylpyridinium, 1-methyl-2-ethylpyridinium, 1,2-diethylpyridinium, 1-(1-butyl)-2-ethylpyridinium, 1-(1-hexyl)-2-ethylpyridinium, 1-(1-octyl)-2-ethylpyridinium, 1-(1-dodecyl)-2-ethylpyridinium, 9-(1-tetradecyl)-2-ethylpyridinium, 1-(1-hexadecyl)-2-ethylpyridinium, 1,2-dimethyl-5-ethylpyridinium, 1,5-diethyl-2-methylpyridinium, 1-(1-butyl)-2-methyl-3-ethylpyridinium, 1-(1-hexyl)-2-methyl-3-ethylpyridinium and 1-(1-octyl)-2-methyl-3-ethylpyridinium, 1-(1-dodecyl)-2-methyl-3-ethylpyridinium, 1-(1-tetradecyl)-2-methyl-3-ethylpyridinium and 1-(1-hexadecyl)-2-methyl-3-ethylpyridinium.

Particularly preferred pyridazinium ions (IVb) are those in which

the radicals R¹ to R⁴ are each hydrogen, or
one of the radicals R¹ to R⁴ is methyl or ethyl and the remaining radicals R¹ to R⁴ are each hydrogen.

Particularly preferred pyrimidinium ions (IVc) are those in which

R¹ is hydrogen, methyl or ethyl and R² to R⁴ are each, independently of one another, hydrogen or methyl, or
R¹ is hydrogen, methyl or ethyl and R² and R⁴ are each methyl and R³ is hydrogen.

Particularly preferred pyrazinium ions (IVd) are those in which

R¹ is hydrogen, methyl or ethyl and R² to R⁴ are each, independently of one another, hydrogen or methyl, or

R¹ is hydrogen, methyl or ethyl and R² and R⁴ are each methyl and R³ is hydrogen, or
R¹ to R⁴ are each methyl or
R¹ to R⁴ are each hydrogen.

Particularly preferred imidazolium ions (IVe) are those in which

R¹ is hydrogen, methyl, ethyl, 1-propyl, 1-butyl, 1-pentyl, 1-hexyl, 1-octyl, 2-hydroxyethyl or 2-cyanoethyl and R² to R⁴ are each, independently of one another, hydrogen, methyl or ethyl.

Particularly useful imidazolium ions (IVe) are 1-methylimidazolium, 1-ethylimidazolium, 1-(1-propyl)imidazolium, 1-(1-allyl)imidazolium, 1-(1-butyl)imidazolium, 1-(1-octyl)-imidazolium, 1-(1-dodecyl)imidazolium, 1-(1-tetradecyl)imidazolium, 1-(1-hexadecyl)-imidazolium, 1,3-dimethylimidazolium, 1,3-diethylimidazolium, 1-ethyl-3-methylimidazolium, 1-(1-butyl)-3-methylimidazolium, 1-(1-butyl)-3-ethylimidazolium, 1-(1-hexyl)-3-methylimidazolium, 1-(1-hexyl)-3-ethylimidazolium, 1-(1-hexyl)-3-butylimidazolium, 1-(1-octyl)-3-methylimidazolium, 1-(1-octyl)-3-ethylimidazolium, 1-(1-octyl)-3-butylimidazolium, 1-(1-dodecyl)-3-methylimidazolium, 1-(1-dodecyl)-3-ethylimidazolium, 1-(1-dodecyl)-3-butylimidazolium, 1-(1-dodecyl)-3-octylimidazolium, 1-(1-tetradecyl)-3-methylimidazolium, 1-(1-tetradecyl)-3-ethylimidazolium, 1-(1-tetradecyl)-3-butylimidazolium, 1-(1-tetradecyl)-3-octylimidazolium, 1-(1-hexadecyl)-3-methylimidazolium, 1-(1-hexadecyl)-3-ethylimidazolium, 1-(1-hexadecyl)-3-butylimidazolium, 1-(1-hexadecyl)-3-octylimidazolium, 1,2-dimethylimidazolium, 1,2,3-trimethylimidazolium, 1-ethyl-2,3-dimethylimidazolium, 1-(1-butyl)-2,3-dimethylimidazolium, 1-(1-hexyl)-2,3-dimethylimidazolium, 1-(1-octyl)-2,3-dimethylimidazolium, 1,4-dimethylimidazolium, 1,3,4-trimethylimidazolium, 1,4-dimethyl-3-ethylimidazolium, 3-methylimidazolium, 3-ethylimidazolium, 3-n-propylimidazolium, 3-n-butylimidazolium, 1,4-dimethyl-3-octylimidazolium, 1,4,5-trimethylimidazolium, 1,3,4,5-tetramethylimidazolium, 1,4,5-trimethyl-3-ethylimidazolium, 1,4,5-trimethyl-3-butylimidazolium, 1,4,5-trimethyl-3-octylimidazolium, 1-prop-1-en-3-yl-3-methylimidazolium and 1-prop-1-en-3-yl-3-butylimidazolium. Especially useful imidazolium ions (IVe) are 1,3-diethylimidazolium, 1-ethyl-3-methylimidazolium, 1-(n-butyl)-3-methylimidazolium.

Particularly preferred pyrazolium ions (IVf), (IVg) and (IVg′) are those in which

R¹ is hydrogen, methyl or ethyl and R² to R⁴ are each, independently of one another, hydrogen or methyl.

Particularly preferred pyrazolium ions (IVh) are those in which R¹ to R⁴ are each, independently of one another hydrogen or methyl.

As particularly preferred pyrazolium ions, mention may be made of pyrazolium and 1,4-dimethylpyrazolium.

1-Pyrazolinium ions (IVi) which are particularly preferably used in the process of the invention are those in which

R¹ to R⁶ are each, independently of one another, hydrogen or methyl.

Particularly preferred 2-pyrazolinium ions (IVj) and (IVj′) are those in which

R¹ is hydrogen, methyl, ethyl or phenyl and R² to R⁶ are each, independently of one another, hydrogen or methyl.

Particularly preferred 3-pyrazolinium ions (IVk) and (IVk′) are those in which

R¹ and R² are each, independently of one another, hydrogen, methyl, ethyl or phenyl and R³ to R⁶ are each, independently of one another, hydrogen or methyl.

Particularly preferred imidazolinium ions (IVl) are those in which

R¹ and R² are each, independently of one another, hydrogen, methyl, ethyl, 1-butyl or phenyl and R³ and R⁴ are each, independently of one another, hydrogen, methyl or ethyl and R⁵ and R⁶ are each, independently of one another, hydrogen or methyl.

Particularly preferred imidazolinium ions (IVm) and (IVm′) are those in which

R¹ and R² are each, independently of one another, hydrogen, methyl or ethyl and R³ to R⁶ are each, independently of one another, hydrogen or methyl.

Particularly preferred imidazolinium ions (IVn) and (IVn′) are those in which

R¹ to R³ are each, independently of one another, hydrogen, methyl or ethyl and R⁴ to R⁶ are each, independently of one another, hydrogen or methyl.

Particularly preferred thiazolium ions (IVo) and (IVo′) and oxazolium ions (IVp) are those in which

R¹ is hydrogen, methyl, ethyl or phenyl and R² and R³ are each, independently of one another, hydrogen or methyl.

In the process according to the invention, particularly preferred 1,2,4-triazolium ions (IVq), (IVq′) and (IVq″) are those in which

R¹ and R² are each, independently of one another, hydrogen, methyl, ethyl or phenyl and R³ is hydrogen, methyl or phenyl.

Particularly preferred 1,2,3-triazolium ions (IVr), (IVr′) and (IVr″) are those in which

R¹ is hydrogen, methyl or ethyl, R² and R³ are each, independently of one another, hydrogen or methyl or R² and R³ are together 1,4-buta-1,3-dienylene.

Particularly preferred pyrrolidinium ions (IVs) are those in which

R¹ is hydrogen, methyl, ethyl or phenyl and R² to R⁹ are each, independently of one another, hydrogen or methyl.

Particularly preferred imidazolidinium ions (IVt) are those in which

R¹ and R⁴ are each, independently of one another, hydrogen, methyl, ethyl or phenyl and R², R³ and R⁵ to R⁸ are each, independently of one another, hydrogen or methyl.

Particularly preferred ammonium ions (IVu) are those in which

R¹ to R³ are each, independently of one another, C₁-C₁₈-alkyl, or
R¹ and R² are together 1,5-pentylene or 3-oxa-1,5-pentylene and R³ is selected from among C₁-C₁₈-alkyl, 2-hydroxyethyl and 2-cyanoethyl.

Examples of tertiary amines from which the quaternary ammonium ions of the general formula (IVu) are derived by quaternization with the abovementioned radical R are diethyl-n-butylamine, diethyl-tert-butylamine, diethyl-n-pentylamine, diethylhexylamine, diethyloctylamine, diethyl-(2-ethylhexyl)amine, di-n-propylbutylamine, din-propyl-n-pentylamine, di-n-propylhexylamine, di-n-propyloctylamine, di-n-propyl-(2-ethylhexyl)amine, diisopropylethylamine, diisopropyl-n-propylamine, diisopropyl-butylamine, diisopropylpentylamine, diisopropylhexylamine, diisopropyloctylamine, diisopropyl-(2-ethylhexyl)amine, di-n-butylethylamine, di-n-butyl-n-propylamine, di-n-butyl-n-pentylamine, di-n-butylhexylamine, di-n-butyloctylamine, di-n-butyl-(2-ethylhexyl)amine, N-n-butylpyrrolidine, N-sec-butylpyrrolidine, N-tert-butylpyrrolidine, N-n-pentylpyrrolidine, N,N-dimethylcyclohexylamine, N,N-diethylcyclohexylamine, N,N-di-n-butylcyclohexylamine, N-n-propylpiperidine, N-isopropylpiperidine, N-n-butyl-piperidine, N-sec-butylpiperidine, N-tert-butylpiperidine, N-n-pentylpiperidine, N-n-butylmorpholine, N-sec-butylmorpholine, N-tert-butylmorpholine, N-n-pentylmorpholine, N-benzyl-N-ethylaniline, N-benzyl-N-n-propylaniline, N-benzyl-N-isopropylaniline, N-benzyl-N-n-butylaniline, N,N-dimethyl-p-toluidine, N,N-diethyl-p-toluidine, N,N-di-n-butyl-p-toluidine, diethylbenzylamine, di-n-propylbenzylamine, di-n-butylbenzylamine, diethylphenylamine, di-n-propylphenylamine and di-n-butylphenylamine.

Preferred tertiary amines (IVu) are diisopropylethylamine, diethyl-tert-butylamine, diisopropylbutylamine, di-n-butyl-n-pentylamine, N,N-di-n-butylcyclohexylamine and tertiary amines derived from pentyl isomers.

Particularly preferred tertiary amines are di-n-butyl-n-pentylamine and tertiary amines derived from pentyl isomers. A further preferred tertiary amine which has three identical radicals is triallylamine.

Particularly preferred guanidinium ions (IVv) are those in which

R¹ to R⁵ are each methyl. A very particularly preferred guanidinium ion (IVv) is N,N,N′,N′,N″,N″-hexamethylguanidinium.

Very particularly preferred cholinium ions (IVw) are those in which

R¹ and R² are each, independently of one another, methyl, ethyl, 1-butyl or 1-octyl and
R³ is hydrogen, methyl, ethyl, acetyl, —SO₂OH or —PO(OH)₂, or
R¹ is methyl, ethyl, 1-butyl or 1-octyl, R² is a —CH₂—CH₂—OR⁴ group and R³ and R⁴ are each, independently of one another, hydrogen, methyl, ethyl, acetyl, —SO₂₀H or —PO(OH)₂, or
R¹ is a —CH₂—CH₂—OR⁴ group, R² is a —CH₂—CH₂—OR⁵ group and R³ to R⁵ are each, independently of one another, hydrogen, methyl, ethyl, acetyl, —SO₂OH or —PO(OH)₂.

As particularly preferred cholinium ions (IVw), mention may be made of those in which R³ is selected from among hydrogen, methyl, ethyl, acetyl, 5-methoxy-3-oxapentyl, 8-methoxy-3,6-dioxaoctyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-oxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl or 14-ethoxy-5,10-oxatetradecyl.

The cations (IV.x.1) are particularly preferably selected from among cations of 1,5-diazabicyclo[4.3.0]non-5-ene (DBN) and 1,8-diazabicyclo[5.4.0]undec-7-ene (DBU).

Particularly preferred phosphonium ions (IVy) are those in which

R¹ to R³ are each, independently of one another, C₁-C₁₈-alkyl, in particular butyl, isobutyl, 1-hexyl or 1-octyl, or phenyl which is unsubstituted or bears 1, 2, 3, 4 or 5 substituents selected independently from among C₁-C₁₈-alkyl, carboxylate, sulfonate, COON and SO₃H.

Particularly preferred sulfonium ions (IVz) are those in which

R¹ and R² are each, independently of one another, C₁-C₁₈-alkyl, in particular butyl, isobutyl, 1-hexyl or 1-octyl.

Among the abovementioned heterocyclic cations, the imidazolium ions, imidazolinium ions, pyridinium ions, pyrazolinium ions and pyrazolium ions are preferred. Particular preference is given to the imidazolium ions and cations of DBU and DBN.

As anions, it is in principle possible to use all polyatomic anions, i.e. multiatomic anions (anions having two or more atoms).

The anion [Y]ⁿ⁻ of the ionic liquid is, for example, selected from the group of pseudohalides and halogen-comprising compounds of the formulae:

BF₄⁻, PF₆⁻, CF₃SO₃⁻, (CF₃SO₃)₂N⁻, CF₃CO₂⁻, CCl₃CO₂⁻, CN⁻, SCN⁻, OCN⁻;
the group of sulfates, sulfites and sulfonates of the general formulae:
SO₄²⁻, HSO₄⁻, SO₃²⁻, HSO₃⁻, R^{c}OSO₃⁻, R^{c}SO₃⁻;
the group of phosphates of the general formulae:
PO₄³⁻, HPO₄²⁻, H₂PO₄⁻, R^{c}PO₄²⁻, HR^{c}PO₄⁻, R^{c}R^{d}PO₄⁻;
the group of phosphonates and phosphinates of the general formulae:

### R^{c}HPO₃⁻, R^{c}R^{d}PO₂⁻, R^{c}R^{d}PO₃⁻;

the group of phosphites of the general formulae:
PO₃³⁻, HPO₃²⁻, H₂PO₃⁻, R^{c}PO₃²⁻, R^{c}HPO₃⁻, R^{c}R^{d}PO₃⁻;
the group of phosphonites and phosphinites of the general formulae:
R^{c}R^{d}PO₂, R^{c}HPO₂⁻, R^{c}R^{d}PO⁻, R^{c}HPO⁻;
the group of carboxylic acids of the general formula:

### R^{c}COO⁻;

anions of hydroxycarboxylic acids and sugar acids;
saccharinates (salts of o-benzoic sulfimide);
the group of borates of the general formulae:
BO₃³⁻, HBO₃²⁻, H₂BO₃⁻, R^{c}R^{d}BO₃⁻, R^{c}HBO₃⁻, R^{c}BO₃²⁻, B(OR^{c})(OR^{d})(OR^{e})(OR^{f})⁻, B(HSO₄)₄⁻, B(R^{c}SO₄)₄⁻;
the group of boronates of the general formulae:

### R^{c}BO₂²⁻, R^{c}R^{d}BO⁻;

the group of carbonates and carbonic esters of the general formulae:

### HCO₃⁻, CO₃²⁻, R^{c}CO₃⁻;

the group of silicates and salicic esters of the general formulae:
SiO₄⁴⁻, HSiO₄³⁻, H₂SiO₄²⁻, H₃SiO₄⁻, R^{c}SiO₄³⁻, R^{c}R^{d}SiO₄²⁻, R^{c}R^{d}R^{e}SiO₄⁻, HR^{c}SiO₄²⁻, H₂R_{c}SiO₄⁻, HR^{c}R^{d}SiO₄⁻;
the group of alkylsilanolates and arylsilanolates of the general formulae:
R^{c}SiO₃³⁻, R^{c}R^{d}SiO₂²⁻, R^{c}R^{d}R^{e}SiO⁻, R^{c}R^{d}R^{e}SiO₃⁻, R^{c}R^{d}R^{e}SiO₂⁻, R^{c}R^{d}SiO₃²⁻;
the group of carboxylmides, bis(sulfonyl)imides and sulfonylimides of the general formulae:

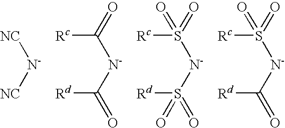

the group of methides of the general formula:

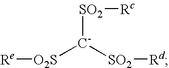

the group of alkoxides and aryloxides of the general formula R^{c}O⁻;
the group of hydrogensulfides, polysulfides, hydrogenpolysulfides and thiolates of the general formulae:

### HS⁻, [Sᵥ]²⁻, [HSᵥ]⁻, [R^{c}S]⁻,

where v is a positive integer from 2 to 10.

Preference is given to the radicals R^{c}, R^{d}, R^{e} and R^{f} each being, independently of one another,
hydrogen;
alkyl, preferably C₁-C₃₀-alkyl, particularly preferably C₁-C₁₈-alkyl, which is unsubstituted or substituted as defined above and/or may be interrupted as defined above by at least one heteroatom or heteroatom-comprising group;
aryl, preferably C₆-C₁₄-aryl, particularly preferably C₆-C₁₀-aryl, which is unsubstituted or substituted as defined above;
cycloalkyl, preferably C₅-C₁₂-cycloalkyl, which is unsubstituted or substituted as defined above;
heterocycloalkyl, preferably heterocycloalkyl having 5 or 6 ring atoms, in which the ring has 1, 2 or 3 heteroatoms or heteroatom-comprising groups in addition to ring carbons and which is unsubstituted or substituted as defined above;
heteroaryl, preferably heteroaryl having from 5 to 10 ring atoms, in which the ring has 1, 2 or 3 heteroatoms or heteroatom-comprising groups selected from among oxygen, nitrogen, sulfur and NR^{a} in addition to ring carbons and which is unsubstituted or substituted as defined above;

where in anions having a plurality of radicals R^{c} to R^{f} two of these radicals together with the part of the anion to which they are bound can form at least one saturated, unsaturated or aromatic ring or ring system which has from 1 to 12 carbon atoms and can have from 1 to 5 nonadjacent heteroatoms or heteroatom-comprising groups which are preferably selected from among oxygen, nitrogen, sulfur and NR^{a} and is unsubstituted or may be substituted.

As regards suitable and preferred C₁-C₃₀-alkyls, in particular C₁-C₁₈-alkyls, C₆-C₁₄-aryls, in particular C₆-C₁₀-aryls, C₅-C₁₂-cycloalkyls, heterocycloalkyls having 5 or 6 ring atoms and heteroaryls having 5 or 6 ring atoms, what has been said above is incorporated by reference at this point. As regards suitable and preferred substituents on C₁-C₃₀-alkyl, especially C₁-C₁₈-alkyl, C₆-C₁₄-aryl, C₅-C₁₂-cycloalkyl, heterocycloalkyl having 5 or 6 ring atoms and heteroaryl having 5 or 6 ring atoms, what has been said above about substituents is likewise incorporated by reference at this point.

When at least one of the radicals R^{c} to R^{f} is optionally substituted C₁-C₁₈-alkyl, then it is preferably methyl, ethyl, propyl, isopropyl, n-butyl, sec-butyl, tert-butyl, pentyl, hexyl, heptyl, octyl, 2-ethylhexyl, 2,4,4-trimethylpentyl, decyl, dodecyl, tetradecyl, hetadecyl, octadecyl, 1,1-dimethylpropyl, 1,1-dimethylbutyl, 1,1,3,3-tetramethylbutyl, benzyl, 1-phenylethyl, α,α-dimethylbenzyl, benzhydryl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonyl-propyl, 1,2-di(methoxycarbonyl)ethyl, 2-methoxyethyl, 2-ethoxyethyl, 2-butoxyethyl, diethoxymethyl, diethoxyethyl, 1,3-dioxolan-2-yl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyl-oxyethyl, chloromethyl, trichloromethyl, trifluoromethyl, 1,1-dimethyl-2-chloroethyl, 2-methoxyisopropyl, 2-ethoxyethyl, butylthiomethyl, 2-dodecylthioethyl, 2-phenylthio-ethyl, 2,2,2-trifluoroethyl, 2-hydroxyethyl, 2-hydroxypropyl, 3-hydroxypropyl, 4-hydroxy-butyl, 6-hydroxyhexyl, 2-aminoethyl, 2-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylamino-propyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl or 6-ethoxyhexyl.

When at least one of the radicals R^{c} to R^{f} is C₁-C₁₈-alkyl interrupted by one or more nonadjacent heteroatoms or heteroatom-comprising groups, then it is preferably 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapenta-decyl, 9-hydroxy-5-oxanonyl, 14-hydroxy-5,10-oxatetradecyl, 5-methoxy-3-oxapentyl, 8-methoxy-3,6-dioxaoctyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-oxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl or 14-ethoxy-5,10-oxatetradecyl.

If two radicals R^{c} to R^{f} form a ring, these radicals can, for example, together form, as fused-on building block, 1,3-propylene, 1,4-butylene, 2-oxa-1,3-propylene, 1-oxa-1,3-propylene, 2-oxa-1,3-propenylene, 1-aza-1,3-propenylene, 1-C₁-C₄-alkyl-1-aza-1,3-propenylene, 1,4-buta-1,3-dienylene, 1-aza-1,4-buta-1,3-dienylene or 2-aza-1,4-buta-1,3-dienylene.

The number of nonadjacent heteroatoms or heteroatom-comprising groups in the radicals R^{c} to R^{f} is in principle not critical and will in general be restricted only by the size of the respective radical or cyclic building block. In general, there will be no more than 5 in the respective radical, preferably no more than 4 or very particularly preferably no more than 3. Furthermore, there is generally at least one carbon atom, preferably at least two carbon atoms, between each two heteroatoms.

Substituted and unsubstituted imino groups can be, for example, imino, methylimino, isopropylimino, n-butylimino or tert-butylimino.

Preferred functional groups of the radicals R^{c} to R^{f} are carboxy, carboxamide, hydroxy, di-(C₁-C₄-alkyl)amino, C₁-C₄-alkyloxycarbonyl, cyano or C₁-C₄-alkoxy. Radicals R^{c} to R^{f} which are different from alkyl can also be substituted by one or more C₁-C₄-alkyl, preferably methyl, ethyl, propyl, isopropyl, n-butyl, sec-butyl or tert-butyl, groups.

When at least one of the radicals R^{c} to R^{f} is optionally substituted C₆-C₁₄-aryl, then it is preferably phenyl, methylphenyl(tolyl), xylyl, α-naphthyl, β-naphthyl, chlorophenyl, dichlorophenyl, trichlorophenyl, difluorophenyl, dimethylphenyl, trimethylphenyl, ethyl-phenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethyl-phenyl, 2,6-dimethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxymethylphenyl.

When at least one of the radicals R^{c} to R^{f} is optionally substituted C₅-C₁₂-cycloalkyl, then it is preferably cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl or a saturated or unsaturated bicyclic system such as norbornyl or norbornenyl.

When at least one of the radicals R^{c} to R^{f} is an optionally substituted five- or six-membered heterocycle, then it is preferably furyl, thienyl, pyryl, pyridyl, indolyl, benzoxazolyl, dioxolyl, dioxyl, benzimidazolyl, benzothiazolyl, dimethylpyridyl, methylquinolyl, dimethylpyrryl, methoxyfuryl, dimethoxypyridyl, difluoropyridyl, methylthiophenyl, isopropylthiophenyl or tert-butylthiophenyl.

When, in anions which have a plurality of radicals R^{c} to R^{f} two of these radicals together with the part of the anion to which they are bound can form at least one saturated, unsaturated or aromatic ring or ring system which has from 1 to 12 carbon atoms and can have from 1 to 5 nonadjacent heteroatoms or heteroatom-comprising groups which are preferably selected from among oxygen, nitrogen, sulfur and NR^{a}, then the ring or ring system is unsubstituted or bears 1, 2, 3, 4, 5 or more than 5 substituents. The substituents are preferably selected independently from among alkyl, alkoxy, alkylsulfanyl, cycloalkyl, cycloalkoxy, polycyclyl, heterocycloalkyl, aryl, aryloxy, arylthio and heteroaryl.

Preferred anions are, for example, selected from the group of pseudohalides and halogen-comprising compounds, the group of carboxylic acids, the group of sulfates, sulfites and sulfonates and the group of phosphates.

Preferred anions are formate, acetate, propionate, butyrate, lactate, saccharinate, carbonate, hydrogencarbonate, sulfate, sulfite, C₁-C₄-alkylsulfates, methanesulfonate, tosylate, trifluoroacetate, C₁-C₄-dialkylphosphates and hydrogensulfate.

Particularly preferred anions are HCOO⁻, CH₃COO⁻, CH₃CH₂COO⁻, carbonate, hydrogencarbonate, sulfate, sulfite, tosylate, CH₃SO₃⁻ or CH₃OSO₃⁻.

Suitable ionic liquids for use in the process of the invention are commercially available, e.g. under the trade name Basionic® from BASF Aktiengesellschaft. Examples of commercially available ionic liquids which can be advantageously used in the process of the invention are 1-ethyl-3-methylimidazolium methanesulfonate (EMIM CH₃SO₃, Basionic ST 35), 1-butyl-3-methylimidazolium methanesulfonate (BMIM CH₃SO₃, Basionic ST 78), methylimidazolium hydrogensulfate (HMIM HSO₄ Basionic AC 39), 1-ethyl-3-methylimidazolium hydrogensulfate (EMIM HSO₄ Basionic AC 25), 1-butyl-3-methylimidazolium hydrogensulfate (BMIM HSO₄ Basionic AC 28) 1-ethyl-3-methylimidazolium acetate (EMIM Acetat, Basionic BC 01), 1-butyl-3-methylimidazolium acetate (BMIM Acetat, Basionic BC 02).

Particular preference is given to 1-ethyl-3-methylimidazolium acetate, 1,3-diethylimidazolium acetate and 1-butyl-3-methylimidazolium acetate.

Cations and anions are present in the ionic liquid. Within the ionic liquid, a proton or an alkyl radical is transferred from the cation to the anion. This forms two uncharged molecules. There is thus an equilibrium in which anions, cations and the two uncharged molecules formed therefrom are present.

The lignocellulose-comprising starting material used according to the invention is, for example, selected from among materials comprising wood fibers and/or other plant fibers. Suitable lignocellulose materials are, for example, the various types of wood such as maple, birch, pear, oak, alder, ash, eucalyptus, hornbeam, cherry, lime, nut tree, poplar, willow, Douglas fir, spruce, yew, hemlock, pine, larch, fir, cedar, etc. Further suitable lignocellulose materials are residues from the wood-processing industry, e.g. wood scrap, sawdust, parquetry grinding dust, etc. Further suitable lignocellulose materials are residues from agriculture, e.g. from the harvesting of cereal (straw), maize, sugar cane (bagasse), etc. Further suitable lignocellulose materials are residues from forestry, e.g. in the form of branches, bark, wood chips, etc. Lignocellulose-comprising starting materials which are preferably used in the process of the invention are the abovementioned cellulose-rich natural fiber materials such as flax, hemp, sisal, jute, straw, coconut fibers, switchgrass (Panicum virgatum) and other natural fibers.

It can be advantageous to subject the lignocellulose-comprising starting material to at least one pretreatment step before or during the treatment with the ionic liquid. Such steps include, for example, mechanical comminution of the cellulose-comprising starting material, e.g. by grinding and/or shredding. In a specific embodiment, the mechanical comminution is carried out in the presence of the ionic liquid. It is advantageous to comminute the lignocellulose material to particles having an average size of not more than 1 cm, preferably not more than 5 mm, in particular not more than 1 mm. If appropriate, further comminution to particles having an average size of not more than 100 μm can be carried out. Owing to their materials properties, fibrous materials (such as flax, hemp, sisal, jute, straw, coconut fibers, switchgrass, etc.) are preferably not subjected to a pressure-shear comminution but to an impact comminution. Suitable milling apparatuses are hammer mills, milling apparatuses operating according to the principle of jet milling and beater mills. The latter are especially suitable for high throughputs.

A suitable process for comminuting fibrous materials (such as flax, hemp, sisal, jute, straw, coconut fibers, switchgrass, etc.) comprises the following steps:
if appropriate, removal of solids such as sand and stones by means of gravity separators and sieving,
if appropriate, precomminution,
comminution in an impact mill, preferably a beater mill,
isolation of the milled material.

The milling of wood is very similar to that of straw. A suitable process for comminuting wood comprises the following steps:
if appropriate, precomminution of the tree branches (in two stages),
comminution in an impact mill, preferably a beater mill,
isolation of the milled material.

Suitable liquid treatment media for carrying out the treatment of the lignocellulose-comprising starting material comprise at least one ionic liquid as defined above.

The treatment of the lignocellulose-comprising starting material with a liquid treatment medium comprising an ionic liquid is generally carried out by bringing the lignocellulose material into intimate contact with the treatment medium. Here, the lignocellulose-comprising starting material is preferably essentially completely solubilized in the treatment medium comprising the ionic liquid. It is advantageously not necessary to subject the solubilized lignocellulose material to a purification step in order to remove insoluble constituents. To carry out the solubilization, the lignocellulose material and the ionic liquid can be brought into intimate contact with one another by customary methods. Suitable apparatuses for this are the customary mixing apparatuses such as stirred vessels and stirred tanks, the abovementioned mechanical comminution apparatuses, etc.

The process of the invention preferably comprises the treatment of the lignocellulose material with at least one ionic liquid as defined above at a temperature of not more than 200° C., particularly preferably not more than 150° C. and in particular not more than 120° C. The treatment is preferably carried out at a temperature of at least 20° C., particularly preferably at least 40° C. Heating can be carried out indirectly or directly, preferably indirectly. For direct heating, it is possible to use a hot heat transfer fluid which is compatible with the ionic liquid used. Indirect heating can be carried out using apparatuses suitable for this purpose, e.g. by means of heat exchangers, heating baths or irradiation with microwaves.

The pressure in the treatment of the lignocellulose material with at least one ionic liquid is generally in the range from 0.1 bar to 100 bar, preferably from 1 bar to 10 bar. In a specific embodiment, the treatment is carried out at ambient pressure.

The duration of the treatment of the lignocellulose material with the ionic liquid is generally from 0.5 minutes to 7 days, preferably from 5 minutes to 96 hours.

The process of the invention advantageously allows treatment of the lignocellulose-comprising starting material with an ionic liquid which comprises additional liquid components in an amount at which no precipitation of solubilized lignocellulose constituents from the treatment medium occurs. Additional liquid components are the precipitants and washing media described in more detail below. Water can, for example, originate from the cellulose-comprising starting material or be present in the ionic liquid (e.g. when the treatment medium comprises ionic liquids recovered from one of the process steps described below). The tolerance of the ionic liquids based on polyatomic anions which are used according to the invention to water represents a significant process simplification, since the additional technical complication associated with working in the absence of water, e.g. for treatment of the lignocellulose under a protective gas atmosphere, costly drying of recovered ionic liquid to remove traces of water, etc., can be dispensed with.

The water content of the liquid treatment medium is preferably from 0.1 to 15% by weight, particularly preferably from 0.5 to 10% by weight, based on the weight of the total treatment liquid (ionic liquid, water and possibly further components which are liquid under the treatment conditions). It is naturally also possible to work at water contents below 0.5% by weight, since the lower limit of the water content for carrying out the process is in principle not critical, while excessively high water contents result in precipitation of the cellulose. The water can originate from the ionic liquid used (for example water which has not been separated off from recovered ionic liquid after the precipitation of cellulose) and from the cellulose material used.

The liquid treatment medium can comprise at least one organic solvent in place of or in addition to water. Suitable organic solvents are those described below as precipitants. The content of organic solvents in the treatment medium is preferably not more than 15% by weight, in particular not more than 10% by weight, based on the total weight of the liquid treatment medium.

The treatment of the lignocellulose material with at least one ionic liquid of the general formula I generally gives a liquid phase comprising cellulose, hemicellulose and lignin in dissolved form. According to the invention, a cellulose-enriched material is isolated from the lignocellulose material which has been treated with the ionic liquid before enzymatic hydrolysis. Isolation is generally effected by addition of a precipitant (P1) and subsequent separation into a cellulose-enriched fraction and a cellulose-depleted fraction (i.e. a first liquid output (O1)). The first precipitant is preferably chosen so that separation into a cellulose-enriched fraction and a lignin-enriched fraction (=first liquid output (O1)) occurs. For this purpose, a solvent or solvent mixture which in combination with the ionic liquid is capable of dissolving lignin is used as precipitant (P1).

As first precipitant (P1), preference is given to using a solvent or solvent mixture selected from among water, alcohols such as methanol, ethanol, n-propanol, isopropanol, n-butanol, tert-butanol, diols and polyols such as ethanediol and propanediol, amino alcohols such as ethanolamine, diethanolamine and triethanolamine, aromatic solvents, e.g. benzene, toluene, ethylbenzene or xylenes, halogenated solvents, e.g. dichloromethane, chloroform, carbon tetrachloride, dichloroethane or chlorobenzene, aliphatic solvents, e.g. pentane, hexane, heptane, octane, ligroin, petroleum ether, cyclohexane and decalin, ethers, e.g. tetrahydrofuran, diethyl ether, methyl tert-butyl ether and diethylene glycol monomethyl ether, ketones such as acetone and methyl ethyl ketone, esters, e.g. ethyl acetate, formamide, dimethylformamide (DMF), dimethylacetamide, dimethyl sulfoxide (DMSO), acetonitrile and mixtures thereof.

The first precipitant (P1) is preferably selected from among organic solvents or solvent mixtures which are at least partially miscible with the ionic liquid used for the treatment of the lignocellulose material. The first precipitant (P1) is particularly preferably completely miscible with the ionic liquid. Preferred organic solvents are the abovementioned alcohols and ketones. Particular preference is given to using at least one alcohol, if appropriate in combination with at least one ketone (this also applies especially when 1-ethyl-3-methylimidazolium acetate or 1,3-diethylimidazolium acetate is used as ionic liquid). The first precipitant (P1) is particularly preferably selected from among methanol, ethanol and mixtures thereof.

The first precipitant (P1) can further comprise ionic liquids. The proportion of ionic liquid in the precipitant will generally be not more than 50% by weight, based on the total weight of the precipitant. Such a content of ionic liquids is not critical to the success of the cellulose precipitation. This ionic liquid comprised in the precipitant can, for example, originate from a use of recovered precipitant, as described below.

The separation into a cellulose-enriched fraction and a liquid fraction (the first liquid output (O1)) is effected, for example by filtration. To accelerate the filtration, it can be carried out under increased pressure on the cellulose side or reduced pressure on the exit side. The separation can likewise be effected by centrifugation. Customary centrifugation processes are described, for example, in G. Hultsch, H. Wilkesmann, “Filtering Centrifuges,” in D. B. Purchas, Solid-Liquid Separation, Upland Press, Croydon 1977, pp. 493-559; and by H. Trawinski in “Die äquivalente Klärfläche von Zentrifugen, Chem. Ztg. 83 (1959), 606-612. Various construction types such as tube and basket centrifuges and especially pusher centrifuges, inverting filter centrifuges and plate separators can be used.

The liquid output (O1) comprising ionic liquid, lignin and precipitant (P1) which is obtained in the separation is preferably subjected to a further fractionation. In particular, a fraction (IL1) comprising essentially the ionic liquid is isolated here. It is in this way possible to recover most of the valuable ionic liquid. In a preferred embodiment of the process of the invention, the liquid output (O1) is subjected to a fractionation to give a fraction (OL1) comprising essentially the ionic liquid, a fraction (Lig 1) comprising essentially the lignin and a fraction (P1) comprising essentially the first precipitant. For example, at least part of the first precipitant (P1) can firstly be separated off from the liquid output (O1) by evaporation. Suitable separation apparatuses are the distillation columns and evaporators customary for this purpose, e.g. falling film evaporators, forced circulation flash evaporators, short path evaporators or thin film evaporators. Owing to the low volatility of the ionic liquids and of the lignin, it is generally possible to dispense with complicated apparatuses as are used in the separation of mixtures having boiling points close together, e.g. complicated column internals, columns having a large number of theoretical plates, etc.

The fraction comprising essentially the first precipitant (P1) can be reused for the separation of the lignocellulose-comprising starting material which has been treated with the ionic liquid into a cellulose-enriched fraction and a cellulose-depleted (lignin-enriched) fraction. This is particularly advantageous when an organic solvent or solvent mixture, e.g. an alcohol, ketone or alcohol/ketone mixture, is used as precipitate.

The composition comprising ionic liquid and lignin which remains after the separation of at least part of the first precipitant (P1) from the liquid output (O1) is preferably subjected to a further fractionation. Here, it is generally not critical if the remaining composition still additionally comprises small amounts of the first precipitant (P1). The further fractionation to give a fraction (IL1) comprising essentially the ionic liquid and a fraction (Lig1) comprising essentially the lignin can be achieved, for example, by extraction or by precipitation of the lignin by means of a further precipitant (P2).

The extraction can be carried out using a solvent which is immiscible with the ionic liquid or at least one solvent which has a miscibility gap with the ionic liquid, in which lignin is sufficiently soluble. The extractant is then brought into intimate contact with the ionic liquid and a phase separation is subsequently carried out.

The further fraction of the liquid output (O1) to give a fraction (IL1) comprising essentially the ionic liquid and a fraction (Lig1) comprising essentially the lignin is preferably carried out by precipitation of the lignin with a second precipitant (P2). (P2) is preferably miscible with the fraction (IL2). Suitable precipitants (P2) are, for example, water; esters, e.g. ethyl acetate; ethers, e.g. tetrahydrofuran, diethyl ether, methyl tert-butyl ether and diethylene glycol monomethyl ether; aliphatic solvents, e.g. pentane, hexane, heptane, octane, ligroin, petroleum ether, cyclohexane and decalin. Preference is given to using water as second precipitant (P2).

The separation into a fraction (Lig1) comprising the precipitated lignin and a fraction (IL1) comprising essentially the ionic liquid is carried out by, for example, filtration or centrifugation. Suitable filtration and centrifugation processes are those described above. The lignin obtained serves, for example, as a source of aromatics. Owing to its high joule value, lignin can also be passed to thermal utilization.

The fractionation of the second liquid phase obtained in the lignin precipitation to give a fraction (IL1) comprising essentially the ionic liquid and a fraction (P2) comprising essentially the second precipitant can be carried out as described above for the first precipitant (P1), preferably by evaporation.

When water is used as second precipitant, it is, owing to the above-described tolerance of the ionic liquids used according to the invention to water, generally not necessary to subject the fraction (IL1) comprising the ionic liquid to an additional removal of the residual water content.

The above-described fractionation of the liquid output (O1) generally enables at least 80% by weight, particularly preferably at least 90% by weight, in particular at least 93% by weight, of the ionic liquid used in the treatment of the lignocellulose-comprising starting material to be recovered. The fraction (IL1) comprising essentially the ionic liquid is preferably reused for the treatment of the lignocellulose-comprising starting material.

As indicated above, it has surprisingly been found that the cellulose material used for enzymatic hydrolysis can still comprise amounts of hemicellulose and/or lignin without the enzymatic hydrolysis being appreciably impaired. Furthermore, it has surprisingly been found that the cellulose material can still comprise amounts of ionic liquid and/or the precipitant (P1) without the enzymatic hydrolysis being appreciably impaired. Thus, it is generally possible for the cellulose-enriched material obtained from the lignocellulose-comprising starting material which has been treated with an ionic liquid to be subjected directly to enzymatic hydrolysis without further work-up. However, to achieve the desired objective of closed materials circuits, it is advantageous to subject the cellulose-enriched material to a further work-up before the enzymatic hydrolysis. The further work-up serves, in particular, to remove ionic liquid still comprised.

For this purpose, the cellulose-enriched material can, for example, be subjected to washing with a liquid washing medium. Suitable washing media are ones in which the ionic liquid readily dissolves and cellulose does not dissolve or dissolves only in small amounts. Preferred washing media are the above-described precipitants (P1). The washing medium is particularly preferably selected from among water and mixtures of water and at least one other water-miscible solvent. Particular preference is given to using water as washing medium.

The treatment of the cellulose-enriched material with a washing medium is preferably carried out at elevated temperature. This is preferably at or below the boiling point of the washing medium. The treatment of the cellulose-enriched material with a washing medium is preferably carried out at a temperature of at least 40° C., particularly preferably at least 60° C., in particular at least 80° C. When water is used as washing medium, the treatment of the cellulose-enriched material is preferably carried out at a temperature of at least 80° C., particularly preferably at least 90° C., in particular at least 95° C.

To remove the ionic liquid comprised, the precipitated cellulose can be subjected to a treatment or a plurality of successive treatments with a washing medium. For this purpose, the cellulose is brought into intimate contact with the washing medium in a suitable apparatus and the washing medium is subsequently separated off from the cellulose. Suitable apparatuses are, for example, stirred vessels which, if necessary, can be provided with a heating facility and a facility for condensation and recirculation of the washing medium. The separation of cellulose and washing medium is effected, for example, by filtration of centrifugation. To accelerate the filtration, it can be carried out under superatmospheric pressure on the cellulose side or reduced pressure on the exit side.

The treatment of the precipitated cellulose to remove ionic liquid still comprised produces a liquid washing medium loaded with ionic liquid (the second liquid output (O2)). The loaded washing medium generally has a content of ionic liquid of from 0.5 to 20% by weight, preferably from 1 to 10% by weight, based on the total weight of the washing medium. In addition, the second liquid output can comprise further components, especially the first precipitant (P1).

The liquid output (O2) can be subjected to a fractionation to give a fraction (IL2) comprising essentially the ionic liquid and a fraction comprising essentially the washing medium and possibly the first precipitant (P1). The ionic liquid can then be reused for the treatment of the lignocellulose-comprising starting material. The washing medium can likewise be reused. If desired, the liquid output (O2) can, depending on its composition, be subjected to a further separation to give at least one of the following fractions
a fraction which comprises essentially the first precipitant (P1) and can, for example, be reused as precipitant,
a water-comprising fraction which can, for example, be reused as washing medium.

In a preferred embodiment of the process of the invention, at least one organic solvent is used as precipitant (P1) and the loaded washing medium is subjected to a separation into
a fraction (IL2) comprising essentially the removed ionic liquid,
a fraction comprising essentially the precipitant (P1) and
a water-comprising fraction.

The lignocellulose material which has been treated with the ionic liquid generally comprises no or only little crystalline material. The content of crystalline material can be determined, for example, by means of X-ray diffraction (XRD) via the ratio of sharp signals to X-ray-amorphous regions.

It has surprisingly been found that cellulose which has been pretreated by the process of the invention can be subjected to rapid enzymatic hydrolysis.

The lignocellulose-comprising starting material which has been treated by the process of the invention is subsequently subjected to an enzymatic hydrolysis.

Suitable enzyme for use in the process of the invention are the cellulases (1,4-(1,3; 1,4)-β-D-glucan-4-glucanohydrolases), which belong to the category of hydrolases. The EC number is 3.2.1.4., and the CAS number is 9012-54-8. The cellulase enzyme complex comprises three different types of enzyme: endoglucanases break the bonds within the cellulose chains, exoglucanases cleave smaller oligosaccharide units, in general disaccharide and tetrasaccharide units (cellobiose, cellotetrose units), from the ends of the smaller chains produced by the endoglucanase. Cellobiases or β-glucosidases cleave the bond between the glucose molecules in the oligosaccharides. Suitable enzymes are, for example, cellulases from Trichoderma reesei (ATCC#26799), which are commercially available from Worthington Biochemical Corporation. Also suitable are the cellulase mixtures, Celluclast 1.5 L with Novozym 188 (Novozymes, Denmark) or Spezyme CP (Genencor International Inc., Rochester, USA) with Novozym 188 (Novozymes, Denmark).

The enzymatic hydrolysis is preferably carried out in an aqueous medium. The aqueous medium used is preferably essentially free of ionic liquids. For the purposes of the present patent application “essentially free of ionic liquids” means a content of less than 0.1% by volume, preferably less than 0.05% by volume, based on the total volume of the liquid reaction medium used for the hydrolysis. The aqueous medium used for the enzymatic hydrolysis is essentially free of ionic liquids as a result of the high degree of recirculation of the ionic liquid achieved by the process of the invention. This is not a stringent requirement for the enzymatic hydrolysis.

The enzymatic hydrolysis is carried out at a pH suitable for the enzyme used. An advantageous pH range for many of the enzymes which can be used according to the invention is from about 4 to 5.5. It is naturally also possible to work at a higher or lower pH in individual cases, as long as the enzyme used permits this. The pH can be set by means of the customary buffer systems known to those skilled in the art. These include acetate buffers, tris-buffers, phosphate buffers, etc.

The enzymatic hydrolysis is preferably carried out at a temperature of from 0 to 80° C., particularly preferably from 20 to 60° C.

In a preferred embodiment of the process of the invention, the materials streams and/or energy flows are integrated so that the ionic liquid used is essentially completely recycled and/or the quantity of heat required in the process (e.g. for the separation of ionic liquid and precipitant) is at least partly used in another step of the process.

A preferred process comprises the following steps:
a) treatment of the lignocellulose-comprising starting material with a liquid treatment medium comprising an ionic liquid, the starting material being solubilized in the treatment medium,
b) precipitation of the cellulose from the solubilizate obtained in step a) by addition of a first precipitant (P1) which in combination with the ionic liquid is capable of dissolving lignin,
c) separation into a cellulose-enriched fraction and a first liquid output (O1) which is enriched in lignin,
d) separation of the output (O1) into a fraction (IL1) comprising essentially the ionic liquid, a fraction (Lig1) comprising essentially the lignin and a fraction comprising essentially the precipitant (P1), with (IL1) being recirculated at least partly to step a) and (F1) being recirculated at least partly to step b),
e) treatment of the cellulose-enriched fraction to remove ionic liquid still comprised and precipitant (P1) possibly still comprised with an aqueous washing medium,
f) separation into a purified cellulose-enriched fraction and a second liquid output (O2),
g) separation of the output (O2) into
   a fraction (IL2) which comprises essentially the removed ionic liquid and is at least partly recirculated to step a),
   a fraction which comprises essentially the precipitant (P1) and is at least partly recirculated to step b),
   a water-comprising fraction which is at least partly recirculated to step e),
h) use of the cellulose-enriched fraction obtained in step f) in the enzymatic hydrolysis.

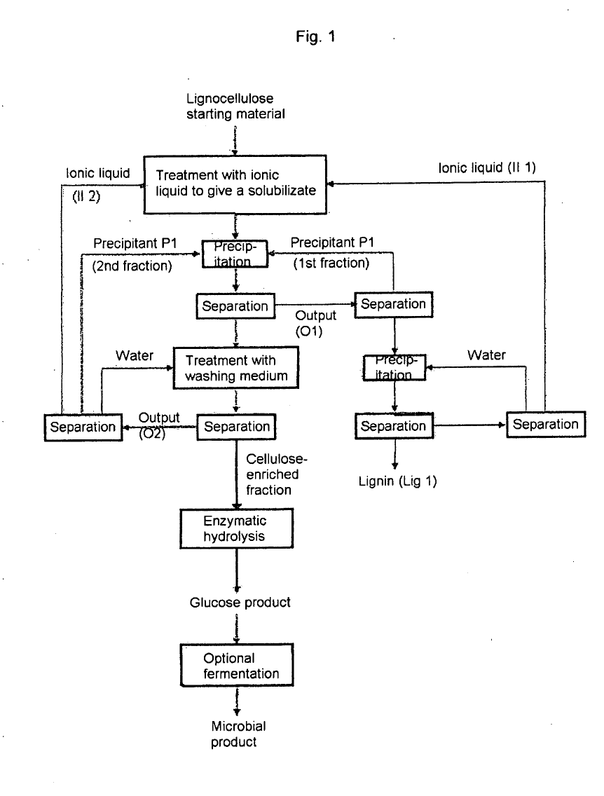

The above-described process is shown schematically in FIG. 1.

As regards suitable and preferred embodiments of steps a) to h), what has been said above about these steps is incorporated by reference. To carry out the separation of the output (O1) in step d), preference is given to firstly separating off at least part of the precipitant (P1) by evaporation, adding a second precipitant (P2) to the composition remaining after (P1) has been separated off, the lignin being at least partly precipitated, and subsequently carrying out a separation into a fraction (Lig1) comprising essentially the lignin and a fraction (IL1) comprising essentially the ionic liquid. The second precipitant (P2) is preferably water; esters, e.g. ethyl acetate; ethers, e.g. tetrahydrofuran, diethyl ether, methyl tert-butyl ether and diethylene glycol monomethyl ether; aliphatic solvents, e.g. pentane, hexane, heptane, octane, ligroin, petroleum ether, cyclohexane and decalin. The second precipitant (P2) is particularly preferably water.

Step h) produces a glucose product which can comprise not only glucose but also components of the lignocellulose-comprising starting material originally used. These include hemicellulose which like glucose is made up of glycosidically linked sugar units but in which the chains are more or less branched and the degree of polymerization is lower than in the case of cellulose (generally from about 50 to 250). Owing to the chemical similarity of hemicellulose and cellulose, the cellulose-enriched material obtained by the process of the invention generally also comprises part of the hemicellulose comprised in the starting material.

In general, the glucose product obtained in step h) comprises not more than 50% by weight, for example not more than 40% by weight, of hemicellulose, based on the total weight of the glucose product.

In a specific embodiment of the process of the invention, enzymes which are also capable of degrading hemicellulose are used for the enzymatic hydrolysis (step h). In this way, it is possible to reduce the hemicellulose content of the glucose product obtained in step h) and at the same time increase the glucose sugar content. The enzymatic hydrolysis of hemicellulose gives mainly arabinose and xylose. Suitable enzymes are the hemicellulases known for this purpose, e.g. xylanases.

The glucose product obtained in step h) generally comprises not more than 30% by weight of lignin, based on the total weight of the glucose product.

In many cases, the glucose product obtained in step h) is suitable for use in a subsequent process, e.g. in a fermentation, without further work-up. In another embodiment, a glucose product which is obtained in step h) and still comprises hemicellulose and/or lignin is subjected to a separation into a fraction comprising essentially the glucose and a fraction comprising hemicellulose and/or lignin (=step i). Here, the glucose-comprising fraction preferably comprises at least 80% by weight, particularly preferably at least 90% by weight, of the glucose comprised in the glucose product. The fraction comprising hemicellulose and/or lignin preferably comprises at least 50% by weight of the lignin comprised in the glucose product and of the hemicellulose.

The glucose-hemicellulose/lignin separation is carried out, for example, by filtration or centrifugation. The above-described processes are suitable for this purpose.

The fraction comprising hemicellulose and/or lignin which is obtained in the optional process step i) can be subjected to a further work-up. If this fraction comprises hemicellulose, it is possible to carry out, for example, an enzymatic hydrolysis using enzymes which are capable of degrading hemicellulose to glucose sugars. In this way, the total amount of glucose sugar obtained in the process of the invention can be increased further. If the lignin content of the fraction comprising hemicellulose and/or lignin is not higher than about 10% by weight, based on the total weight of hemicellulose and lignin, an enzymatic degradation of hemicellulose is possible even without prior removal of lignin. The degradation product obtained in this way can, if desired, be subjected to a fractionation to give a fraction comprising essentially the glucose and further sugards such as arabinose and xylose and a fraction comprising lignin.

The invention further provides the glucose product which can be obtained by the process of the invention. This is, in a first embodiment, the glucose product which can be obtained in step h) and comprises glucose together with components of the lignocellulose-comprising starting material originally used. It is preferably a glucose product which comprises from 0.1 to 50% by weight, particularly preferably from 0.5 to 40% by weight, especially from 1 to 25% by weight, based on the total weight of the glucose product, of hemicellulose. In addition to or in place of hemicellulose, the glucose product can comprise further sugars different from glucose, especially arabinose and xylose. The glucose product preferably comprises not more than 15% by weight, particularly preferably not more than 10% by weight, of lignin, based on the total weight of the glucose product. The lignin content is generally at least 0.001% by weight, for example at least 0.01% by weight, based on the total weight of the glucose product. In a second embodiment, the glucose product of the invention is the glucose product which can be obtained in step i). This preferably comprises at least 80% by weight, particularly preferably at least 90% by weight, of glucose. It is preferably a glucose product which contains from 0.1 to 20% by weight, for example from 0.5 to 10% by weight, based on the total weight of the glucose product, of hemicellulose and/or sugars different from glucose, especially arabinose and xylose. The lignin content is generally at least 0.001% by weight, for example at least 0.01% by weight, based on the total weight of the glucose product.

The invention further provides the lignin product which can be obtained by the process of the invention. In contrast to lignin products known from the prior art, those according to the invention are free of sulfur-comprising compounds.

The separation of glucose and lignin is effected, for example, by filtration or centrifugation. To accelerate the filtration, it can be carried out under superatmospheric pressure on the cellulose side or reduced pressure on the outflow side.

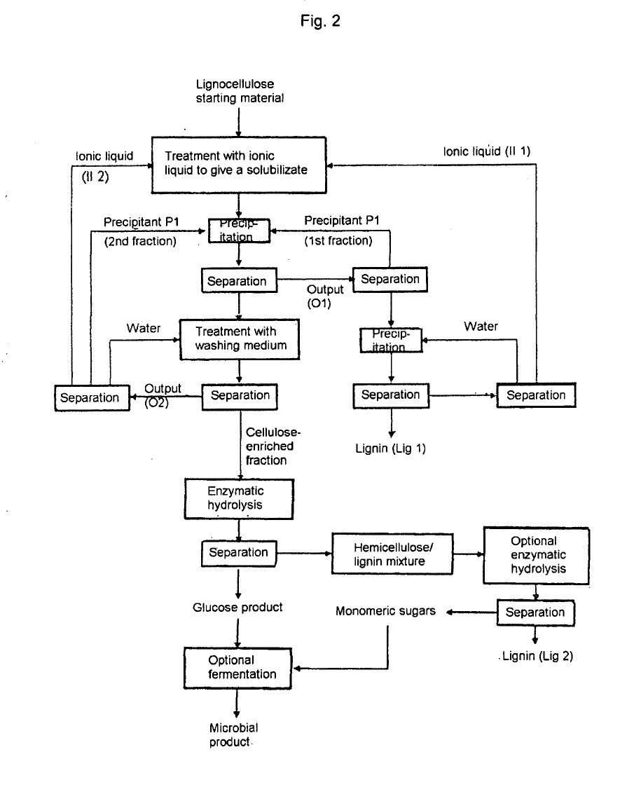

The above-described process is shown schematically in FIG. 2.

Shrinking petroleum reserves and increasing fuel prices are leading to increasing interest in replacing petroleum-based fuels by inexpensive and environmentally friendly alternatives. Processes for producing fuels from biogenic fat- or oil-comprising starting mixtures and used oils and animal fats have been known for some time, with rapeseed oil predominantly being used at present as starting material in the production of biogenic fuels in central Europe. Biogenic oils and fats themselves are less suitable as fuel for engines since they have to be purified beforehand by means of usually complicated processes. A known solution to this problem is to convert the triglycerides comprised in the biogenic oil and fat starting mixtures into monoalkyl esters of fatty acids, in particular methyl or ethyl esters. These esters, which are also referred to as “biodiesel”, can generally be used in diesel engines without great modifications. However, biodiesel is relatively expensive because of the raw material prices and the refining processes required and cannot compete on price with normal diesel fuel. A good supplement would be the use of ethanol as product of the fermentation of glucose. The invention therefore further provides a process for producing a microbial metabolite, in particular ethanol, which additionally comprises the step k):
k) fermentation of the glucose product obtained in step h) or step i).

Sugar-comprising liquid media are a basic starting material for many fermentation processes; the sugars comprised in the media are transformed by the microorganisms used to give organic products of value. Microbial metabolites, i.e. organic compounds which can be obtained in this way, comprise, for example, low molecular weight volatile compounds such as ethanol, nonvolatile metabolites such as amino acids, vitamins and carotenoids and also many further substances. The process of the invention makes it possible to produce volatile and nonvolatile microbial metabolites having at least 2 carbon atoms by fermentation. The glucose which can be obtained by the process of the invention, which can, as mentioned above, comprise small amounts of oligosaccharides, is suitable as starting material.

Microbial metabolites which can be obtained by the process of the invention are, in particular, alcohols, e.g. ethanol, n-propanol, n-butanol, etc.; diols, e.g. ethanediol, propanediol and butanediol; higher-hydric alcohols having 3 or more, e.g. 3, 4, 5 or 6 OH groups, e.g. glycerol, sorbitol, mannitol, xylitol and arabinitol (pentane-1,2,3,4,5-pentol); relatively long-chain monocarboxylic, dicarboxylic and tricarboxylic acids which bear 1 or more, e.g. 1, 2, 3 or 4, hydroxyl groups and preferably from 2 to 10 carbon atoms, e.g. glycolic acid, tartaric acid, itaconic acid, succinic acid, propionic acid, lactic acid, 3-hydroxypropionic acid, fumaric acid, maleic acid, 2,5-furandicarboxylic acid, glutaric acid, levulinic acid, gluconic acid, aconitic acid and citric acid; amino acids, e.g. lysine, glutamic acid, methionine, phenylalanine, aspartic acid, tryptophan and threonine; purine and pyrimidine bases; nucleosides and nucleotides, e.g. nicotinamide adenine dinucleotide (NAD) and adenosine 5′-monophosphate (AMP); lipids; saturated and unsaturated fatty acids having preferably from 10 to 22 carbon atoms, e.g. γ-linolenic acid; vitamins and provitamins, e.g. ascorbic acid, vitamin B₆, vitamin B12 and riboflavin; proteins, e.g. enzymes such as amylases, pectinases, cellulases, esterases such as lipases, pancreases, proteases, xylanases and oxidoreductases such as laccases, catalases and peroxidases, glucanases, phytases; carotenoids, e.g. lycopene, β-carotene, astaxanthin, zeaxanthin and canthaxanthin; ketones having preferably from 3 to 10 carbon atoms and possibly one or more hydroxyl groups, e.g. acetone and acetoin; lactones, e.g. γ-butyrolactone, cyclodextrins, biopolymers, e.g. polyhydroxyacetate, polyesters, e.g. polylactide, polyisoprenoids, polyamides; and also precursors and derivatives of the compounds mentioned. Further microbial metabolites are described by Gutcho in Chemicals by Fermentation, Noyes Data Corporation (1973), ISBN: 0818805086.

In particular, the metabolites produced are selected from among alkanols having from 2 to 10 carbon atoms, alkanediols having from 2 to 10 carbon atoms, enzymes, amino acids, vitamins, aliphatic monocarboxylic and dicarboxylic acids having from 2 to 10 carbon atoms, aliphatic hydroxycarboxylic acids having from 2 to 10 carbon atoms and ketones having from 2 to 10 carbon atoms.

Compounds prepared by a fermentation route are in each case obtained in the enantiomeric form produced by the microorganisms used (if different enantiomers exist). The microorganisms used in the fermentation are chosen in a manner known per se according to the respective microbial metabolites. They can be of natural origin or be genetically modified. Examples of suitable microorganisms and fermentation processes are shown in Table A.

TABLE A

| Material | Microorganism | Reference |
|---|---|---|
| Ethanol | Saccharomyces, | The Alcohol Textbook - A reference for the |
| | Schizosaccharomyces, | beverage, fuel and industrial alcohol industries, |
| | Saccharomycodes, | Jaqus et al. (Ed.), Nottingham Univ. Press |
| | Torulopsis, | 1995, ISBN 1-8977676-735 |
| | Kluyveromyces, | |
| | Zymomonas mobilis, E. coli | |
| Tartaric acid | Lactobacilli, (e.g. | Rehm, H.-J.: Biotechnology, Weinheim, VCH, |
| | Lactobacillus | 1980 and 1993-1995; |
| | delbrueckii) | Gutcho, Chemicals by Fermentation, Noyes |
| | | Data Corporation (1973), |
| Itaconic acid | Aspergillus terreus, | Jakubowska, in Smith & Pateman (Ed.), |
| | Aspergillus itaconicus | Genetics and Physiology of Aspergillus, |
| | | London: Academic Press 1977; Miall, in Rose |
| | | (Ed.), Economic Microbiology, Vol. 2, pp. 47-119, |
| | | London: Academic Press 1978; U.S. Pat. No. |
| | | 3,044,941 (1962). |
| Succinic acid | Actinobacillus sp. | Int. J. Syst. Bacteriol. 26, 498-504 (1976); |
| | 130Z, | EP 249773 (1987), Inv.: Lemme & Datta; U.S. Pat. No. |
| | Anaerobiospirillum | 5,504,004 (1996), Inv.: Guettler, Jain & Soni; |
| | succiniproducens, | Arch. Microbiol. 167, 332-342 (1997); |
| | Actinobacillus | Guettler MV, Rumler D, Jain MK., Actinobacillus |
| | succinogenes, E. coli | succinogenes sp. nov., a novel succinic-acid- |
| | | producing strain from the bovine rumen. Int J |
| | | Syst Bacteriol. 1999 Jan; 49 Pt 1: 207-16; |
| | | U.S. Pat. No. 5,723,322, U.S. Pat. No. 5,573,931, U.S. Pat. No. 5,521,075, |
| | | WO99/06532, U.S. Pat. No. 5,869,301, U.S. Pat. No. 5,770,435 |
| Hydroxy- | Lactobacillus | RÖMPP Online Version 2.2 |
| propionic acid | delbrückii, L. leichmannii or | |
| | Sporolactobacillus | |
| | inulinus | |
| Propionic acid | Propionibacterium, e.g. | Rehm, H.-J.: Biotechnology, Weinheim, VCH, |
| | P. arabinosum, P. schermanii, | 1980 and 1993-1995; |
| | P. freudenreichii, | Gutcho, Chemicals by Fermentation, Noyes |
| | Clostridium | Data Corporation (1973), |
| | propionicum, | |
| Diaminopimelic | Corynebacterium | Rehm, H.-J.: Biotechnology, Weinheim, VCH, |
| acid | glutamicum | 1980 and 1993-1995; |
| | | Gutcho, Chemicals by Fermentation, Noyes |
| | | Data Corporation (1973), |
| Citric acid | Aspergillus niger, | Crit. Rev. Biotechnol. 3, 331-373 (1986); |
| | Aspergillus wentii | Food Biotechnol. 7, 221-234 (1993); 10, 13-7 |
| | | (1996). |
| Aconitic acid | Aspergillus niger, | Crit. Rev. Biotechnol. 3, 331-373 (1986); |
| | Aspergillus wentii | Food Biotechnol. 7, 221-234 (1993); 10, 13-27 |
| | | (1996).; Rehm, H.-J.: Biotechnology, |
| | | Weinheim, VCH, 1980 and 1993-1995; |
| Malic acid | Aspergilli, e.g. | U.S. Pat. No. 3,063,910 |
| | Aspergillus flavus, A. niger, | |
| | A. oryzae, | |
| | Corynebacterium | |
| Gluconic acid | Aspergilli, e.g. A. niger | Gutcho, Chemicals by Fermentation, Noyes |
| | | Data Corporation (1973), |
| Butyric acid | Clostridium (e.g. | Rehm, H.-J.: Biotechnology, Weinheim, VCH, |
| | Clostridium | 1980 and 1993-1995; |
| | acetobutylicum, | |
| | C. butyricum) | |
| Lactic acid | Lactobacillus e.g. L. delbrückii, | Rehm, H.-J.: Biotechnology, Weinheim, VCH, |
| | L. leichmannii, | 1980 and 1993-1995; |
| Lysine | Corynebacterium | Ikeda, M.: Amino Acid Production Process |
| | glutamicum | (2003), Adv. Biochem. Engin/Biotechnol 79, 1-35. |
| Glutamatic acid | Corynebacterium | Ikeda, M.: Amino Acid Production Process |
| | glutamicum | (2003), Adv. Biochem. Engin/Biotechnol 79, 1-35. |
| Methionine | Corynebacterium | Ikeda, M.: Amino Acid Production Process |
| | glutamicum | (2003), Adv. Biochem. Engin/Biotechnol 79, 1-35. |
| Phenylalanine | Corynebacterium | Trends Biotechnol. 3, 64-68 (1985); J. Ferment. |
| | glutamicum, E. coli | Bioeng. 70, 253-260 (1990). |
| Threonine | E. coli | Ikeda, M.: Amino Acid Production Process |
| | | (2003), Adv. Biochem. Engin/Biotechnol 79, 1-35. |
| Aspartic acid | E. coli | Ikeda, M.: Amino Acid Production Process |
| | | (2003), Adv. Biochem. Engin/Biotechnol 79, 1-35+ |
| | | ref. cited there, |
| | | Gutcho, Chemicals by Fermentation, Noyes |
| | | Data Corporation (1973) |
| Purine and | Bacillus subtilis | Rehm, H.-J.: Biotechnology, Weinheim, VCH, |
| pyrimidine | | 1980 and 1993-1995; |
| bases | | Gutcho, Chemicals by Fermentation, Noyes |
| | | Data Corporation (1973), |
| Nicotinamide | Bacillus subtilis | Rehm, H.-J.: Biotechnology, Weinheim, VCH, |
| adenine | | 1980 and 1993-1995; |
| dinucleotide | | Gutcho, Chemicals by Fermentation, Noyes |
| (NAD) | | Data Corporation (1973), |
| Adenosine 5′- | Bacillus subtilis | Rehm, H.-J.: Biotechnology, Weinheim, VCH, |
| monophosphate | | 1980 and 1993-1995; |
| (AMP) | | Gutcho, Chemicals by Fermentation, Noyes |
| | | Data Corporation (1973), |
| γ-Linolenic acid | Mucor, Mortiella, | Gill, I., Rao, V.: Polyunsaturated fatty acids, |
| | Aspergillus spp. | part 1: occurence, biological activities and |
| | | applications (1997). Trends in Biotechnology |
| | | 15 (10), 401-409; Zhu, H.: Utilization of Rice |
| | | Brain by Pythium irregulare for Lipid |
| | | Production. Master Thesis Lousiana State |
| | | University, 31.10.2002 (URN etd-1111102- |
| | | 205855). |
| Dihomo-γ- | Mortiella, Conidiobolus, | Gill, I., Rao, V.: Polyunsaturated fatty acids, |
| linolenic acid | Saprolegnia spp. | part 1: occurence, biological activities and |
| | | applications (1997). Trends in Biotechnology |
| | | 15 (10), 401-409; Zhu, H.: Utilization of Rice |
| | | Brain by Pythium irregulare for Lipid |
| | | Production. Master Thesis Lousiana State |
| | | University, 31.10.2002 (URN etd-1111102- |
| | | 205855). |
| Arachidonic | Mortiella, Phytium spp. | Gill, I., Rao, V.: Polyunsaturated fatty acids, |
| acid | | part 1: occurence, biological activities and |
| | | applications (1997). Trends in Biotechnology |
| | | 15 (10), 401-409; Zhu, H.: Utilization of Rice |
| | | Brain by Pythium irregulare for Lipid |
| | | Production. Master Thesis Lousiana State |
| | | University, 31.10.2002 (URN etd-1111102- |
| | | 205855). |
| Eicosa- | Mortiella, Phytium spp., | Gill, I., Rao, V.: Polyunsaturated fatty acids, |
| pentaenic acid | Rhodopseudomonas, | part 1: occurence, biological activities and |
| | Shewanella spp. | applications (1997). Trends in Biotechnology |
| | | 15 (10), 401-409; Zhu, H.: Utilization of Rice |
| | | Brain by Pythium irregulare for Lipid |
| | | Production. Master Thesis Lousiana State |
| | | University, 31.10.2002 (URN etd-1111102- |
| | | 205855). |
| Docosa- | Thraustochytrium, | Gill, I., Rao, V.: Polyunsaturated fatty acids, |
| hexaenic acid | Entomophthora spp., | part 1: occurence, biological activities and |
| | Rhodopseudomonas, | applications (1997). Trends in Biotechnology |
| | Shewanella spp. | 15 (10), 401-409; Zhu, H.: Utilization of Rice |
| | | Brain by Pythium irregulare for Lipid |
| | | Production. Master Thesis Lousiana State |
| | | University, 31.10.2002 (URN etd-1111102- |
| | | 205855). |
| Propanediol | E. coli | DE 3924423, U.S. Pat. No. 440379, WO 9635799, U.S. Pat. No. |
| | | 5,164,309 |
| Butanediol | Enterobacter | Rehm, H.-J.: Biotechnology, Weinheim, VCH, |
| | aerogenes, Bacillus | 1980 and 1993-1995; |
| | subtilis, Klebsiella | Gutcho, Chemicals by Fermentation, Noyes |
| | oxytoca | Data Corporation (1973); |
| | | H. G. SCHLEGEL and H. W. JANNASCH, |
| | | 1981; |
| | | Afschar et al.: Mikrobielle Produktion von 2,3- |
| | | Butandiol. CIT 64 (6), 2004, 570-571 |
| Butanol | Clostridium (e.g. | Rehm, H.-J.: Biotechnology, Weinheim, VCH, |
| | Clostridium | 1980 and 1993-1995; |
| | acetobutylicum, | Gutcho, Chemicals by Fermentation, Noyes |
| | C. propionicum) | Data Corporation (1973), |
| Glycerol | Yeast, Saccharomyces | Gutcho, Chemicals by Fermentation, Noyes |
| | rouxii | Data Corporation (1973), |
| Mannitol | Aspergillus candida, | Gutcho, Chemicals by Fermentation, Noyes |
| | Torulopsis | Data Corporation (1973), |
| | mannitofaciens | |
| Arabitol | Saccharomyces rouxii, | Gutcho, Chemicals by Fermentation, Noyes |
| | S. mellis, Sclerotium | Data Corporation (1973), |
| | glucanicum, Pichia | |
| | ohmeri | |
| Xylitol | Saccharomyces | Gutcho, Chemicals by Fermentation, Noyes |
| | cerevisiae | Data Corporation (1973), |
| Hyaluronic acid | Streptococcus sp. | Rehm, H.-J.: Biotechnology, Weinheim, VCH, |
| | | 1980 and 1993-1995; |
| Ascorbic acid | Gluconobacter | RÖMPP Online Version 2.2 |
| | melanogenes | |
| Vitamin B₁₂ | Propionibacterium spp., | Chem. Ber. 1994, 923-927; ROMPP Online |
| | Pseudomonas | Version 2.2 |
| | denitrificans | |
| Riboflavin | Bacillus subtilis, | WO 01/011052, DE 19840709, WO 98/29539, |
| | Ashbya gossypii | EP 1186664; Fujioka, K.: New biotechnology |
| | | for riboflavin (vitamin B₂) and character of this |
| | | riboflavin. Fragrance Journal (2003), 31(3), 44-48. |
| Vitamin B₆ | Rhizobium tropici, R. meliloti | EP 0765939 |
| Enzymes | Aspergilli (e.g. | Rehm, H.-J.: Biotechnology, Weinheim, VCH, |
| | Aspergillus niger A. oryzae), | 1980 and 1993-1995; |
| | Trichoderma, | Gutcho, Chemicals by Fermentation, Noyes |
| | E. coli, Hansenulna or | Data Corporation (1973), |
| | Pichia (e.g. Pichia | |
| | pastorius), Bacillus | |
| | (e.g. Bacillus | |
| | licheniformis, B. subtilis) | |
| | and many | |
| | others | |
| Zeaxanthin | Dunaliella salina | Jin et al (2003) Biotech. Bioeng. 81: 115-124 |
| Canthaxanthin | Brevibacterium | Nelis et al (1991) J Appl Bacteriol 70: 181-191 |
| Lycopene | Blakeslea trispora, | WO 03/056028, EP 01/201762, WO 01/12832, |
| | Candida utilis | WO 00/77234, |
| | | Miura et al (1998) Appl Environ Microbiol |
| | | 64: 1226-1229 |
| β-Carotene | Blakeslea trispora, | Kim S., Seo W., Park Y., Enhanced production |
| | Candida utilis | of beta-carotene from Blakeslea trispora with |
| | | Span 20, Biotechnology Letters, Vol 19, No 6, |
| | | 1997, 561-562; Mantouridou F., Roukas T.: |
| | | Effect of the aeration rate and agitation speed |
| | | on beta-carotene production and morphology |
| | | of Blakeslea trispora in a stirred tank reactor: |
| | | mathematical modelling, Biochemical |
| | | Engineering Journal 10 (2002), 123-135; WO |
| | | 93/20183; WO 98/03480, Miura et al (1998) |
| | | Appl Environ Microbiol 64: 1226-1229 |
| Astaxanthin | Phaffia Rhodozyma, | U.S. Pat. No. 5,599,711; WO 91/02060, |
| | Candida utilis | Miura et al (1998) Appl Environ Microbiol |
| | | 64: 1226-1229 |
| Polyhydroxy- | Escherchia coli, | S. Y. Lee, Plastic Bacteria Progress and |
| alkanoates, | Alcaligenes latus, and | Prospects for polyhydroxyalkanoate production |
| polyesters | many others | in bacteria, Tibtech, Vol. 14, (1996), pp. 431-438., |
| | | Steinbüchel, 2003; Steinbüchel (Ed.), |
| | | Biopolymers, 1^{st} edition, 2003, Wiley-VCH, |
| | | Weinheim and references cited there |
| Polyisoprenoids | Lactarius sp., | Steinbüchel (Ed.), Biopolymers, 1^{st} edition, |
| | Hygrophorus sp., | 2003, Wiley-VCH, Weinheim and references |
| | Russula sp. | cited there |
| Acetone | Clostridium (e.g. | Rehm, H.-J.: Biotechnology, Weinheim, VCH, |
| | Clostridium | 1980 and 1993-1995; |
| | acetobutylicum, | Gutcho, Chemicals by Fermentation, Noyes |
| | C. propionicum) | Data Corporation (1973) |
| Acetoin | Enterobacter | Lengeler, J. W., Drews, G., Schlegel, H. G.: Ed., |
| | aerogenes, Clostridium | Biology of the Procaryotes, Thieme, Stuttgart |
| | acetobutylicum, | (1999), p. 307; RÖMPP Online-Edition |
| | Lactococcus lactis | |
| Thurigensin | Bacillus thuringiensis | Jian-Zhong Jong et al.: Fed-batch culture of |
| | | Bacillus thuringiensis for thuringensin |
| | | production in a tower type bioreactor. |
| | | Biotechnology and Bioengineering 48 (3) |
| | | (2004), 207-213. |
| Polyketides | Streptomyces fradiae, | Kirst: Fermentation-derived compounds as a |
| | Sorangium cellulosum | source for new products. Pure & Appl. Chem. |
| | | 70 (2), (1998), 335-338; Zirkle et al.: |
| | | Heterologous production of the antifungal |
| | | polyketide antibiotic soraphen A of Sorangium |
| | | cellulosum So ce26 in Streptomyces lividans. |
| | | Microbiology 150 (8), (2004), 2761-74. |
| Gibberellic acid | Gibberella fujikuroi | Hollmann et al.: Extraktiv-Fermentation von |
| | | Gibberellinsäure mit Gibberella fujikuroi. CIT 7 |
| | | (1995), 892-895. |

In a preferred embodiment, the fermentation is carried out without addition of separate enzymes.

It is also possible to use immobilized microorganisms in the process of the invention for producing a microbial metabolite. To immobilize the microorganisms, they are, for example, mixed with a support protein (e.g. gelatin) and crosslinked by means of glutaraldehyde, embedded in a synthetic polymer, e.g. polyacrylamide or embedded in a natural polymer such as agar, collagen, kappa-carrageenan or alginate. Suitable fermentation vessels are in principle vessels configured in the manner of a bioreactor and are known to those skilled in the art.

In preferred embodiments of the invention, the organic compound produced is ethanol. The fermentation in step k) for producing ethanol can be carried out using the appropriate microorganisms listed in Table A), e.g. as an anaerobic fermentation (alcoholic fermentation). To isolate the ethanol, it can be advantageous firstly to remove the solid constituents from the fermentation broth, e.g. by means of centrifugation or filtration, and subsequently isolate the ethanol from the liquid phase, e.g. by distillation. Customary filtration methods are, for example, cake filtration and deep bed filtration (e.g. as described in A. Rushton, A. S. Ward, R. G. Holdich: Solid-Liquid Filtration and Separation Technology, VCH Verlagsgesellschaft, Weinheim 1996, pp. 177ff., K. J. Ives, in A. Rushton (Ed.): Mathematical Models and Design Methods in Solid-Liquid Separation, NATO ASI series E No. 88, Martinus Nijhoff, Dordrecht 1985, pp. 90ff.) and cross-flow filtrations, in particular microfiltration for removal of solids having a size of >0.1 μm (e.g. as described in J. Altmann, S. Ripperger, J. Membrane Sci. 124 (1997) 119-128). Customary centrifugation methods are described, for example, in G. Hultsch, H. Wilkesmann, “Filtering Centrifuges,” in D. B. Purchas, Solid- Liquid Separation, Upland Press, Croydon 1977, pp. 493-559; and H. Trawinski. The equivalent clearing area of centrifuges is described in Chem. Ztg. 83 (1959) 606-612. The alcohol present in the slurry is distilled by methods customary in the prior art and purified further if appropriate. Known distillation, rectification and dewatering processes can be used.

The invention is illustrated by the following, nonlimiting examples.

Ionic liquids from BASF Aktiengesellschaft were used.

The cellulose activity is determined by the standard filter paper assay and reported as filter paper units per gram of glucane (FPU) (Ghose Tk. 1987, Measurement of cellulase activities. Pure Appl. Chem. 59 (2):257-268).

The lignocellulose material used (poplar wood or switchgrass) is subjected to comminution by milling in an Alpine LU 100 universal rotor mill provided with Ultraplex rotor and screen basket before treatment with anionic liquid. The milled material obtained has a size of less than 300 μm.

### I. Solubilization of the Lignocellulose Material in an Ionic Liquid and Isolation of a Cellulose-Enriched Fraction

### EXAMPLE 1

### Treatment of Poplar Wood with 1-ethyl-3-methylimidazoliumacetate (EMIM Acetate)

107.6 g of EMIM acetate and 5.0 g of poplar wood are stirred at 100° C. for 69 hours. The wood is dissolved well; only fine particles can be seen. 188.6 g (240 ml) of an acetone/ethanol mixture (1:1) are added to the wood solution at 40° C. and the resulting mixture is stirred for another one hour. Filtration under reduced pressure and washing with 70.6 g of an acetone/ethanol mixture gives a cellulose-enriched product.

The cellulose-enriched product is once again boiled in 500 ml of hot water, filtered off with suction and washed twice with about 100 ml of hot water. The moist product obtained in this way can subsequently be used for enzymatic hydrolysis or be dried at 100° C. under reduced pressure to determine the yield (weight obtained=2.96 g).

The mixture of ionic liquid, acetone and ethanol and also constituents of the lignocellulose material still dissolved therein which has been separated off from the cellulose-enriched product is evaporated on a rotary evaporator. This gives 89.6 g of the ionic liquid comprising constituents of the lignocellulose material dissolved therein which are again subjected to precipitation in 600 ml of hot water. This gives a light-brown, turbid suspension which is filtered with suction through a fiberglass filter (weight obtained=0.55 g of lignin).

Analysis: Elemental analysis

### EXAMPLE 2

### Treatment of Switchgrass with EMIM Acetate

105.3 g of EMIM acetate and 5.0 g of milled switchgrass are mixed at room temperature, heated to 100° C. and stirred at this temperature for 69 hours. The fibers are dissolved well; only fine particles are to be seen. 240 ml of an acetone/ethanol:mixture (1:1) are added to the fiber solution at 40° C. and the resulting mixture is stirred for another one hour. Filtration under reduced pressure and washing with 70.6 g of an acetone/ethanol mixture gives a cellulose-enriched product.

The cellulose-enriched product is once again boiled in 500 ml of hot water, filtered off with suction and washed twice with about 100 ml of water. The moist product obtained in this way can subsequently be used for enzymatic hydrolysis or be dried at 100° C. under reduced pressure to determine the yield (weight obtained=2.94 g).

The mixture of ionic liquid, acetone and ethanol and also constituents of the lignocellulose material still dissolved therein which has been separated off from the cellulose-enriched product is evaporated on a rotary evaporator. This gives 90.4 g of the ionic liquid comprising constituents of the lignocellulose material dissolved therein which are again subjected to precipitation in 600 ml of hot water. This gives a light-brown, turbid suspension which is filtered with suction through a fiberglass filter (weight obtained=0.57 g of lignin).

Analysis: Elemental analysis

The mixture of ionic liquid and water which has been separated off from the suspension is evaporated in a falling film evaporator (Sambay evaporator) to recover the ionic liquid.

### EXAMPLE 3

### Treatment of Poplar Wood with 1,3-diethylimidazolium Acetate (EEIM Acetate)

760.0 g of EEIM acetate are mixed with 40.0 g of milled poplar wood at room temperature, the mixture is heated to 100° C. and stirred for 46 hours. The wood is then completely dissolved.

To carry out the precipitation, 3.5 l of ethanol are placed in a vessel at 60° C. and the wood solution is then added slowly. The mixture is stirred at 60° C. for 30 minutes and subsequently cooled while stirring over a period of 30 minutes. The precipitate formed is filtered off with suction over a period of 2 hours and boiled in 3 l of hot water to remove residual ionic liquid. The cellulose-enriched product obtained in this way is once again filtered off with suction.

Weight obtained: 279.6 g (moist)
Dry mass determination: 10.2%
28.5 g (dry, corresponds to a yield of 71.2%)

Analysis: Elemental analysis

The mixture of ionic liquid and ethanol and also constituents of the lignocellulose material still dissolved therein which has been separated off from the cellulose-enriched product is evaporated by means of a falling film evaporator (702.3 g, 92.3%). To precipitate the lignin remaining in the solution, 4 l of hot water are added and the mixture is stirred for 5 hours. After the precipitate has settled, it is slowly filtered off with suction. The mixture of ionic liquid and ethanol is evaporated in a falling film evaporator (Sambay) to recover the ionic liquid.

Apart from the ionic liquids used in Examples 1 to 3, those mentioned below can be used analogously with equal success:
1-butyl-3-methylimidazolium acetate
1-dodecyl-3-methylimidazolium acetate
1-hexadecyl-3-methylimidazolium acetate
1-ethyl-3-methylimidazolium diethylphosphate
1-ethyl-3-methylimidazolium hydrogensulfate
1-ethyl-3-methylimidazolium methanesulfonate
1-ethyl-3-methylimidazolium octanoate
HDBU acetate
methylDBU acetate

### II. Enzymatic Degradation

### EXAMPLE 4

### Enzymatic Degradation of the Cellulose Products from Examples 1 and 2

A cellulose product derived from lignocellulose material obtained from poplar wood (Example 1) or switchgrass (Example 2), in each case obtainable as described above, is suspended in a proportion on a dry basis of 1% for switchgrass and 1.5% for poplar in 0.05 M acetate buffer at a pH of 4.8. In parallel to the lignocellulose preparations which were treated with ionic liquids, milled poplar wood and switchgrass were used without pretreatment with an ionic liquid as comparative examples.

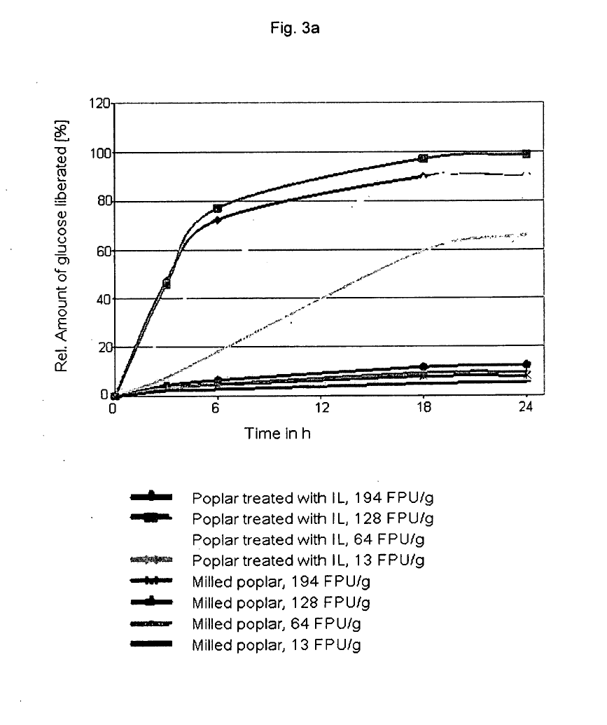

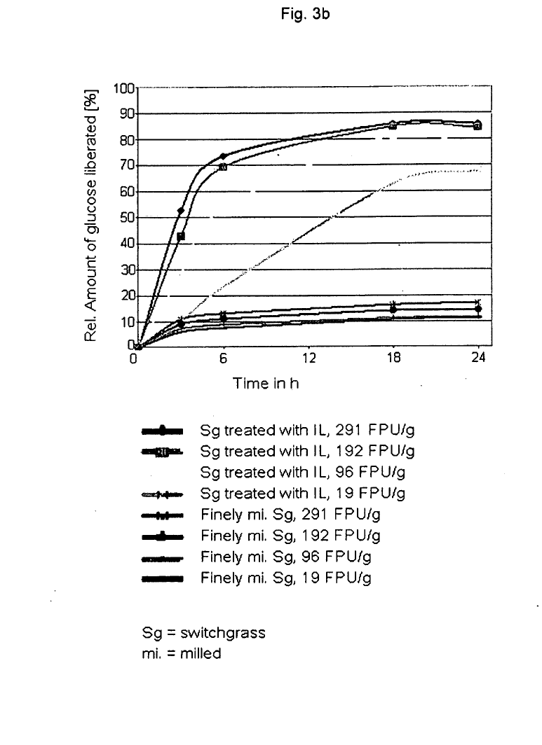

Various amounts of a cellulase mixture, Celluclast 1.5 L (Novozymes, Denmark, 700EG/g) with Novozym 188 (Novozymes, Denmark, 250 CBU/g) are added to all batches in a volumetric ratio of 4:1. The amounts of Celluclast vary in the range from 13 FPU to 291 FPU/g of cellulose product from lignocellulose material, and the amounts of Novozym range from 88 CBU/g of cellulose product from lignocellulose material to 0.34 CBU/g of cellulose product from lignocellulose material. After incubation at 55° C. for 3, 6, 18 and 24 hours, samples are taken in each case. After sampling, the samples are briefly heated to 95° C. to deactivate the enzyme. The samples are then centrifuged, filtered through a 0.22 μm filter and the glucose content is determined by means of HPLC. The measured values for the amount of glucose liberated in the individual samples are shown in FIGS. 3a (poplar) and 3b (switchgrass).

As can be seen from FIG. 3, the proportion of glucose liberated from a sample material which has been pretreated according to the invention with an ionic liquid is significantly increased compared to the liberation of glucose from a material which has not been pretreated. At the same amounts of cellulose-degrading enzymes used, in the case of an untreated lignocellulose material only a maximum of 17% (switchgrass) or 13% (poplar) of the amount of cellulose available for degradation is converted into glucose. As a result of treatment with an ionic liquid, the amount of enzyme can be reduced to 19 FPU/g of cellulose material while still observing a liberation of glucose of 70% of the maximum liberation of glucose. At higher amounts of enzyme, virtually complete conversion of the digestable amount of cellulose can be achieved. In all experiments using pretreated lignocellulose material, the initial hydrolysis rate is a number of times that observed in the case of untreated biomass.

### EXAMPLE 5

### Enzymatic Degradation of the Cellulose Product from Example 3

A cellulose product derived from lignocellulose material obtained from poplar wood, which can be obtained as described above in Example 3 (treatment with EEIM acetate), is suspended in a proportion by weight on a dry basis of 2.31% in 0.05 M acetate buffer at a pH of 4.8. In addition, a cellulose product derived from lignocellulose material obtained from poplar wood, which can be obtained as described in Example 1 (treatment with EEIM acetate), is suspended in a proportion by weight on a dry basis of 2.31% in 0.05 M acetate buffer at a pH 4.8.

Various amounts of a cellulase mixture, Celluclast 1.5 L (Novozymes, Denmark, 700EG/g) with Novozym 188 (Novozymes, Denmark, 250 CBU/g) are added to all batches in a volumetric ratio of 4:1. Optimash BG (Genencor) is used for the degradation of hemicelluloses. The amount of Celluclast varied in the range from 5 FPU to 25 FPU/g of lignocellulose, and the amount of Novozym 188 ranged from 3 CBU/g of lignocellulose to 17.5 CBU/g of lignocellulose and the amounts of Optimash ranged from 0.01% to 1%.

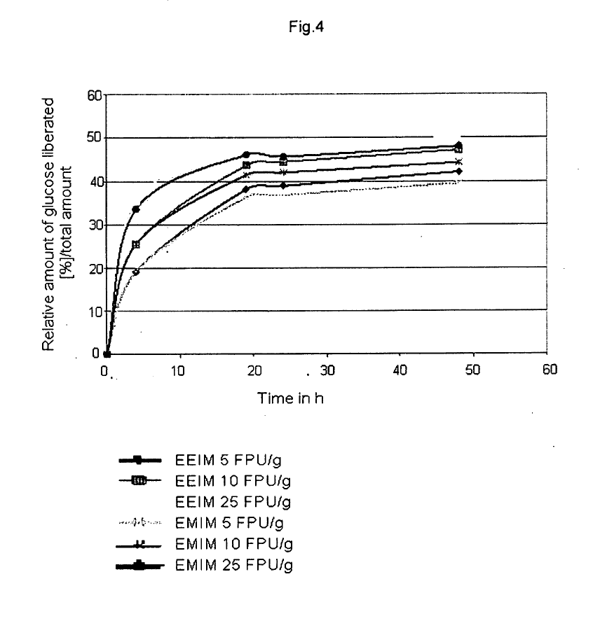

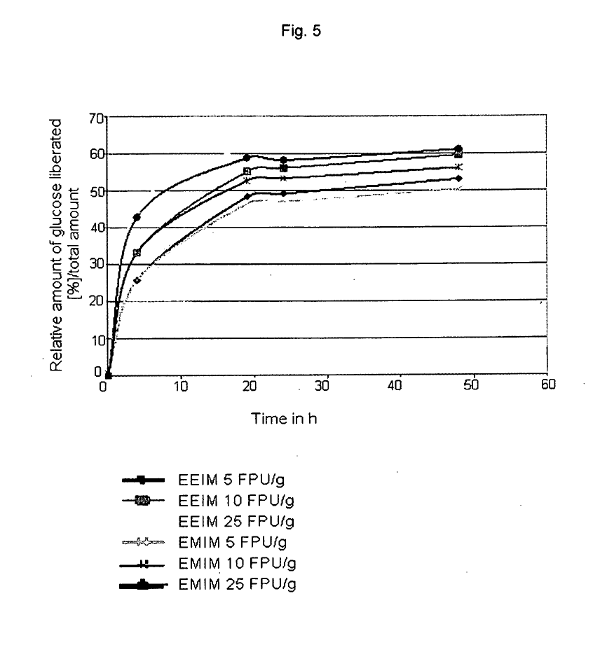

After incubation at 55° C. for 0, 4, 19, 24, and 48 hours, samples were taken in each case. After sampling, the samples were briefly heated to 95° C. to deactivate the enzyme. The samples were then centrifuged off, filtered through a 0.22 μm filter and the glucose content was examined by means of HPLC. The measured values for the relative amount of glucose or xylose liberated based on the amount of lignocellulose material used in the individual samples are shown in graph form in FIGS. 4 and 5.

As can be seen from the figures, the proportion of glucose and xylose liberated from lignocellulose material which has been pretreated according to the invention with the ionic liquids EMIM acetate or EEIM acetate it is comparable, i.e. lignocellulose material treated with EEIM Ac and lignocellulose material treated with EMIM acetate display the same digestability by the enzyme mixture of Celluclast, Novozym 188 and Optimash BG.

1. A process for preparing a glucose product from a lignocellulose material, in which
a lignocellulose-comprising starting material is provided and treated with a liquid treatment medium which comprises an ionic liquid whose anions are selected from among polyatomic anions,
a cellulose-enriched material is isolated from the treated material and
the cellulose-enriched material is subjected to an enzymatic hydrolysis.

2. The process according to claim 1, wherein at least one ionic liquid selected from among
(A) salts of the general formula (I)
[A]ₙ⁺[Y]ⁿ⁻ (I),
where n is 1, 2, 3 or 4, [A]⁺ is a quaternary ammonium cation, an oxonium cation, a sulfonium cation or a phosphonium cation and [Y]ⁿ⁻ is a multiatomic, monovalent, divalent, trivalent or tetravalent anion or a mixture of these anions;
(B) mixed salts of the general formulae (II.a), (II.b) and (II.c)
[A¹]⁺[A²]⁺[Y]ⁿ⁻ (II.a), where n=2,
[A¹]⁺[A²]⁺[A³]⁺[Y]ⁿ⁻ (II.b), where n=3,
[A¹]⁺[A²]⁺[A³]⁺[A⁴]⁺[Y]ⁿ⁻ (II.c), where n=4,
where [A¹]⁺, [A²]⁺, [A³]⁺ and [A⁴]⁺ are selected independently from among the groups mentioned for [A]⁺ and [Y]ⁿ⁻ is as defined under (A); or
(C) mixed salts of the general formulae (III.a) to (III.j)
[A¹]⁺[A²]⁺[A³]⁺[M¹]⁺[Y]ⁿ⁻ (III.a), where n=4,
[A¹]⁺[A²]⁺[M¹]⁺[M²]⁺[Y]ⁿ⁻ (III.b), where n=4,
[A¹]⁺[M¹]⁺[M²]⁺[M³]⁺[Y]ⁿ⁻ (III.c), where n=4,
[A¹]⁺[A²]⁺[M¹]⁺[Y]ⁿ⁻ (III.d), where n=3,
[A¹]⁺[M¹]⁺[M²]⁺[Y]ⁿ⁻ (III.e), where n=3,
[A¹]⁺[M¹]⁺[Y]ⁿ⁻ (III.f), where n=2,
[A¹]⁺[A²]⁺[M⁴]²⁺[Y]ⁿ⁻ (III.g), where n=4,
[A¹]⁺[M¹]⁺[M⁴]²⁺[Y]ⁿ⁻ (III.h), where n=4,
[A¹]⁺[M⁵]³⁺[Y]ⁿ⁻ (III.i), where n=4,
[A¹]⁺[M⁴]²⁺[Y]ⁿ⁻ (III.j), where n=3,
where [A¹]⁺, [A²]⁺ and [A³]⁺ are selected independently from among the groups mentioned for [A]⁺, [Y]ⁿ⁻ is as defined under (A) and [M¹]⁺, [M²]⁺, [M³]⁺ are monovalent metal cations, [M⁴]²⁺ is a divalent metal cation and [M⁵]³⁺ is a trivalent metal cation,
is used.

3. The process according to either claim 1 or 2, wherein at least one ionic liquid having at least one cation selected from among compounds of the formulae (IV.a) to (IV.z), 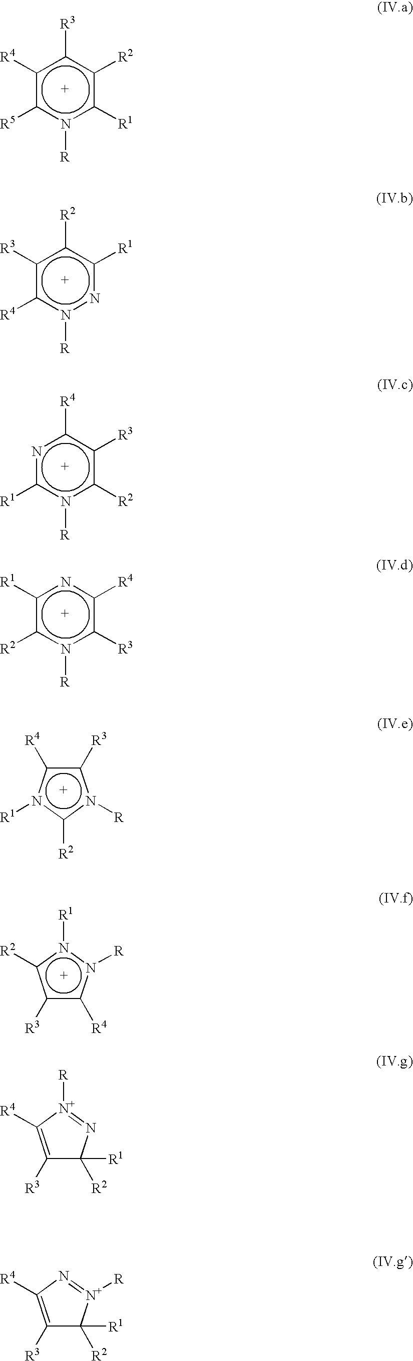 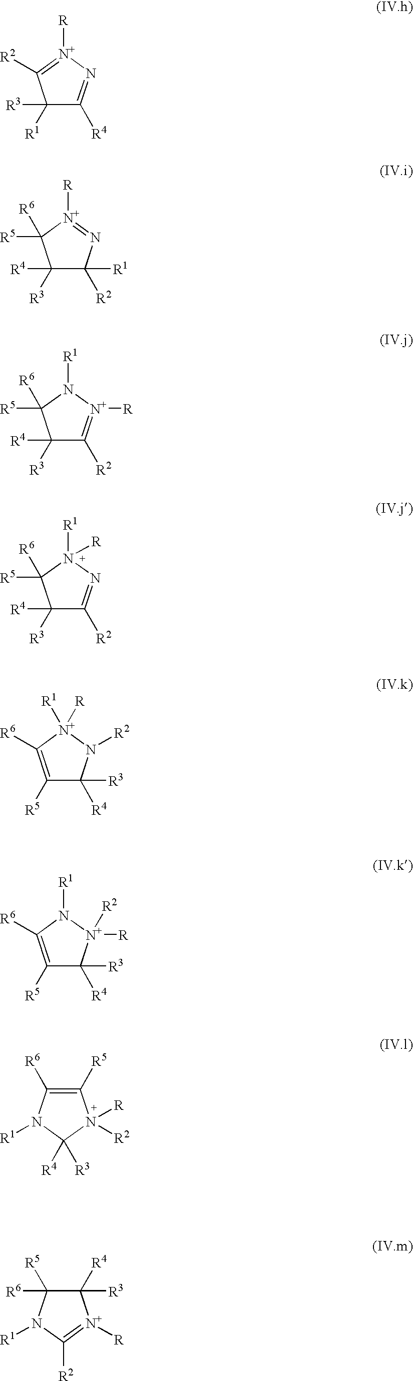 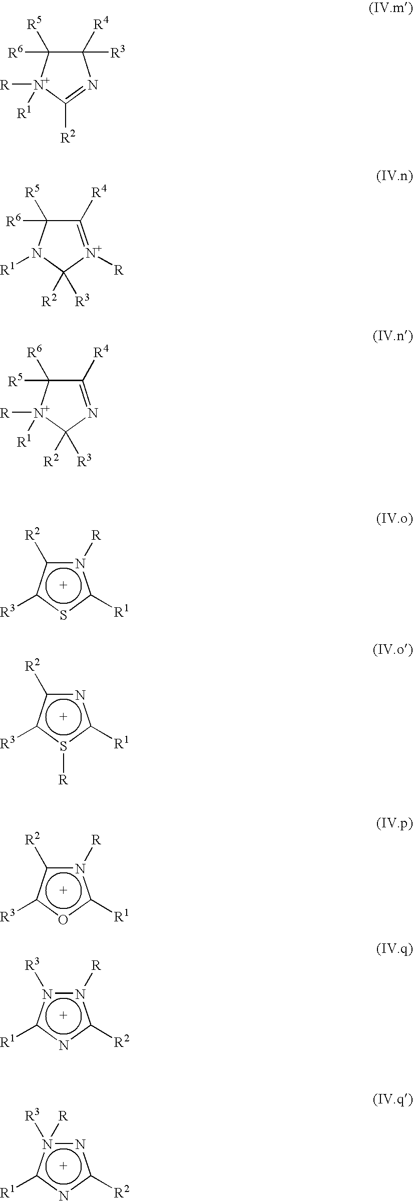 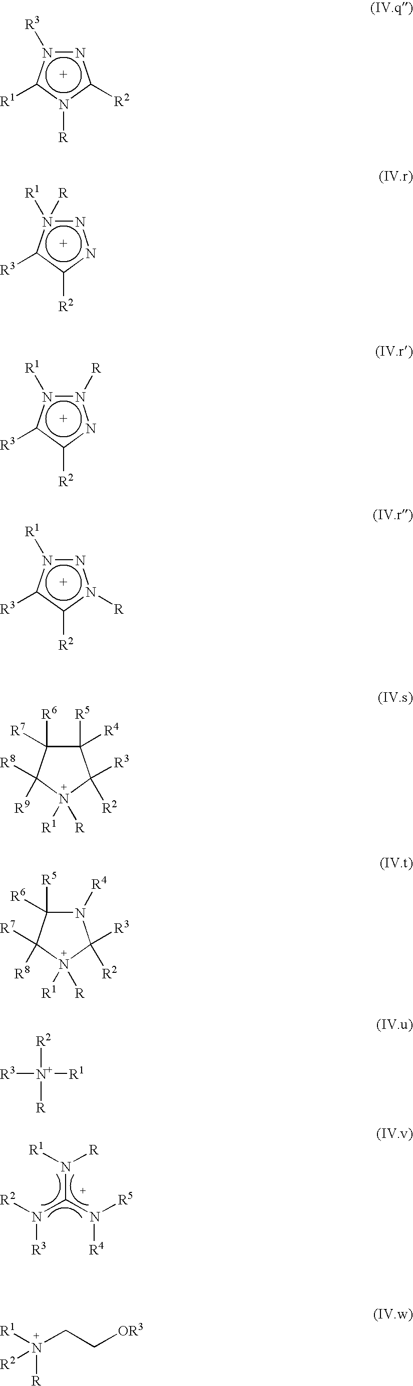 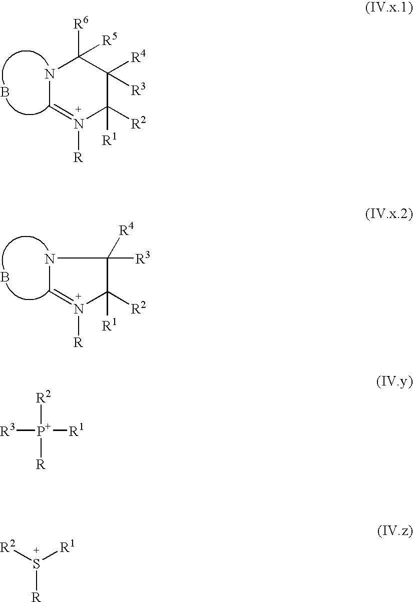
and oligomers comprising these structures, where
R is hydrogen, alkyl, alkenyl, cycloalkyl, cycloalkenyl, polycyclyl, heterocycloalkyl, aryl or heteroaryl;
radicals R¹, R², R³, R⁴, R⁵, R⁶, R⁷, R⁸ and R⁹ which are bound to a ring carbon are each, independently of one another, hydrogen, a sulfo group, COOH, carboxylate, sulfonate, acyl, alkoxycarbonyl, cyano, halogen, hydroxyl, SH, nitro, NE¹E², alkyl, alkoxy, alkylthio, alkylsulfinyl, alkylsulfonyl, alkenyl, cycloalkyl, cycloalkyloxy, cycloalkenyl, cycloalkenyloxy, polycyclyl, polycyclyloxy, heterocycloalkyl, aryl, aryloxy or heteroaryl, where E¹ and E² are each, independently of one another, hydrogen, alkyl, cycloalkyl, heterocycloalkyl, aryl or hetaryl,
radicals R¹, R², R³, R⁴, R⁵, R⁶, R⁷, R⁸ and R⁹ which are bound to a ring heteroatom are each, independently of one another, hydrogen, SO₃H, NE¹E², alkyl, alkoxy, alkenyl, cycloalkyl, cycloalkenyl, polycyclyl, heterocycloalkyl, aryl or heteroaryl, where E¹ and E² are each, independently of one another, hydrogen, alkyl, cycloalkyl, heterocycloalkyl, aryl or hetaryl, or
two adjacent radicals R¹ to R⁹ together with the ring atoms to which they are bound may also form at least one fused, saturated, unsaturated or aromatic ring or ring system which has from 1 to 30 carbon atoms and may comprise from 1 to 5 nonadjacent heteroatoms or heteroatom-comprising groups and be unsubstituted or substituted, and
two geminal radicals R¹ to R⁹ may also together be ═O, ═S or ═NR^{b}, where R^{b} is hydrogen, alkyl, cycloalkyl, aryl or heteroaryl, and
R¹ and R³ or R³ and R⁵ in the compounds of the formula (IV.x.1) may together also be the second part of a double bond between the ring atoms bearing these radicals, and
B in the compounds of the formulae (IV.x.1) and (IV.x.2) together with the C—N group to which it is bound forms a 4- to 8-membered, saturated or unsaturated or aromatic ring which may optionally be substituted and/or may optionally have further heteroatoms or heteroatom-comprising groups and/or may comprise further fused saturated, unsaturated or aromatic carbocycles or heterocycles,
is used.

4. The process according to claim 3, wherein at least one ionic liquid having at least one cation selected from among imidazolium ions of the formula (IV.e) is used.

5. The process according to any of the preceding claims, wherein at least one ionic liquid having at least one anion selected from:
the group of pseudohalides and halogen-comprising compounds of the formulae:
BF₄⁻, PF₆⁻, CF₃SO₃⁻, (CF₃SO₃)₂N⁻, CF₃CO₂⁻, CCl₃CO₂⁻, CN⁻, SCN⁻, OCN⁻;
the group of sulfates, sulfites and sulfonates of the general formulae:
SO₄²⁻, HSO₄⁻, SO₃²⁻, HSO₃⁻, R^{c}OSO₃⁻, R^{c}SO₃⁻;
the group of phosphates of the general formulae:
PO₄³⁻, HPO₄²⁻, H₂PO₄⁻, R^{c}PO₄²⁻, HR^{c}PO₄⁻, R^{c}R^{d}PO₄⁻;
the group of phosphonates and phosphinates of the general formulae:
R^{c}HPO₃⁻,R^{c}R^{d}PO₂⁻, R^{c}R^{d}PO₃⁻;
the group of phosphites of the general formulae:
PO₃³⁻, HPO₃²⁻, H₂PO₃⁻, R^{c}PO₃²⁻, R^{c}HPO₃⁻, R^{c}R^{d}PO₃⁻;
the group of phosphonites and phosphinites of the general formulae:
R^{c}R^{d}PO₂⁻, R^{c}HPO₂⁻, R^{c}R^{d}PO⁻, R^{c}HPO⁻;
the group of carboxylic acids of the general formula:
R^{c}COO⁻;
anions of hydroxycarboxylic acids and sugar acids;
saccharinates (salts of o-benzoic sulfimide);
the group of borates of the general formulae:
BO₃³⁻, HBO₃²⁻, H₂BO₃⁻, R^{c}R^{d}BO₃⁻, R^{c}HBO₃⁻, R^{c}BO₃²⁻,
B(OR^{c})(OR^{d})(OR^{e})(OR^{f})⁻, B(HSO₄)₄⁻, B(R^{c}SO₄)₄⁻;
the group of boronates of the general formulae:
R^{c}BO₂²⁻, R^{c}R^{d}BO⁻;
the group of carbonates and carbonic esters of the general formulae:
HCO₃⁻, CO₃²⁻, R^{c}CO₃⁻;
the group of silicates and salicic esters of the general formulae:
SiO₄⁴⁻, HSiO₄³⁻, H₂SiO₄²⁻, H₃SiO₄⁻, R^{c}SiO₄³⁻, R^{c}R^{d}SiO₄²⁻, R^{c}R^{d}R^{e}SiO₄⁻, HR^{c}SiO₄²⁻, H₂R^{c}SiO₄⁻, HR^{c}R^{d}SiO₄⁻;
the group of alkylsilanolates and arylsilanolates of the general formulae:
R^{c}SiO₃³⁻, R^{c}R^{d}SiO₂²⁻, R^{c}R^{d}R^{e}SiO⁻, R^{c}R^{d}R^{e}SiO₃⁻, R^{c}R^{d}R^{e}SiO₂⁻, R^{c}R^{d}SiO₃²⁻;
the group of carboxylmides, bis(sulfonyl)imides and sulfonylimides of the general formulae: 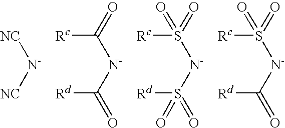
the group of methides of the general formula: 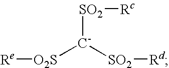
the group of alkoxides and aryloxides of the general formula R^{c}O⁻;
the group of hydrogensulfides, polysulfides, hydrogenpolysulfides and thiolates of the general formulae:
HS⁻, [Sᵥ]²⁻, [HSᵥ]⁻, [R^{c}S]⁻, where v is a positive integer from 2 to 10,
where the radicals R^{c}, R^{d}, R^{e} and R^{f} are selected independently from among hydrogen, alkyl, cycloalkyl, heterocycloalkyl, aryl, heteroaryl,
where in anions having a plurality of radicals R^{c} to R^{f} two of these radicals together with the part of the anion to which they are bound can form at least one saturated, unsaturated or aromatic ring or ring system which has from 1 to 12 carbon atoms and can have from 1 to 5 nonadjacent heteroatoms or heteroatom-comprising groups which are preferably selected from among oxygen, nitrogen, sulfur and NR^{a} and is unsubstituted or may be substituted.

6. The process according to any of the preceding claims, wherein at least one ionic liquid having at least one anion selected from the group of pseudohalides and halogen-comprising compounds, the group of carboxylic acids, the group of sulfates, sulfites and sulfonates or the group of phosphates is used.

7. The process according to any of the preceding claims, wherein the lignocellulose-comprising starting material is subjected to mechanical comminution before or during the treatment with the ionic liquid.

8. The process according to any of the preceding claims, wherein the lignocellulose-comprising starting material is solubilized in the treatment medium comprising the ionic liquid.

9. The process according to any of the preceding claims, wherein the cellulose-enriched material is isolated from the treated material by addition of a precipitant (P1) and subsequent separation into a cellulose-enriched fraction and a cellulose-depleted fraction.

10. The process according to claim 9, wherein a solvent or solvent mixture which in combination with the ionic liquid is capable of dissolving lignin is used as precipitant (P1).

11. The process according to either claim 9 or 10, wherein the precipitant (P1) is selected from among organic solvents or solvent mixtures which are at least partially, preferably completely, miscible with the ionic liquid used for the treatment of the lignocellulose material.

12. The process according to any of claims 9 to 11, wherein the mixture obtained in the precipitation is fractionated to give a cellulose-enriched fraction and a liquid output (O1) which is enriched in lignin.

13. The process according to claim 12, wherein the liquid output (O1) is subjected to a separation into a fraction (IL1) comprising essentially the ionic liquid, a fraction (Lig1) comprising essentially the lignin and a fraction (P1) comprising essentially the precipitant.

14. The process according to claim 13, wherein at least part of the precipitant (P1) is firstly separated off by evaporation, a precipitant (P2) is added to the composition remaining after (P1) has been separated off, resulting in the lignin being at least partly precipitated, and a separation into a fraction (Lig1) comprising essentially the lignin and a fraction (IL1) comprising essentially the ionic liquid is subsequently carried out.

15. The process according to claim 13, wherein the fraction (IL1) comprising essentially the ionic liquid is reused for the treatment of the lignocellulose-comprising starting material.

16. The process according to any of the preceding claims, wherein the cellulose-enriched material is subjected to a treatment to remove ionic liquid still comprised.

17. The process according to claim 16, wherein the cellulose-enriched material is subjected to washing with a liquid washing medium.

18. The process according to claim 17, wherein the treatment of the cellulose-enriched material with a washing medium is carried out at a temperature of at least 40° C., preferably at least 60° C., in particular at least 80° C.

19. The process according to either claim 17 or 18, wherein the washing medium comprises water or consists of water.

20. A process for producing a microbial metabolite having at least two carbon atoms, which comprises fermentation of glucose obtained by a process according to any of claims 1 to 19.

21. The process according to claim 20, wherein the metabolite comprises ethanol.

22. The process according to any of claims 1 to 21 comprising the following steps:
a) treatment of the lignocellulose-comprising starting material with a liquid treatment medium comprising an ionic liquid, the starting material being solubilized in the treatment medium,
b) precipitation of the cellulose from the solubilizate obtained in step a) by addition of a first precipitant (P1) which in combination with the ionic liquid is capable of dissolving lignin,
c) separation into a cellulose-enriched fraction and a first liquid output (O1) which is enriched in lignin,
d) separation of the output (O1) into a fraction (IL1) comprising essentially the ionic liquid, a fraction (Lig1) comprising essentially the lignin and a fraction comprising essentially the precipitant (P1), with (IL1) being recirculated at least partly to step a) and (F1) being recirculated at least partly to step b),
e) treatment of the cellulose-enriched fraction to remove ionic liquid still comprised and precipitant (P1) possibly still comprised with an aqueous washing medium,
f) separation into a purified cellulose-enriched fraction and a second liquid output (O2),
g) separation of the output (O2) into
a fraction (IL2) which comprises essentially the removed ionic liquid and is at least partly recirculated to step a),
a fraction which comprises essentially the precipitant (P1) and is at least partly recirculated to step b),
a water-comprising fraction which is at least partly recirculated to step e),
h) use of the cellulose-enriched fraction obtained in step f) in the enzymatic hydrolysis.

23. The process according to claim 22, wherein, in step d), at least part of the precipitant (P1) is firstly separated off by evaporation, a second precipitant (P2) is added to the composition remaining after (P1) has been separated off, the lignin being at least partly precipitated, and a separation into a fraction (Lig1) comprising essentially the lignin and a fraction (IL1) comprising essentially the ionic liquid is subsequently carried out.

24. The process according to any of the preceding claims, wherein enzymes which are capable of degrading hemicellulose to sugars, especially xylose, are additionally used for the enzymatic hydrolysis.

25. The process according to any of claims 22 to 24, wherein the glucose product obtained in step h) is subjected to a separation into a fraction comprising essentially the glucose and a fraction comprising hemicellulose and/or lignin (=step i).

26. The process according to any of claims 22 to 25 for producing a microbial metabolite having at least two carbon atoms, which additionally comprises
k) fermentation of the glucose product obtained in step h) or step i).

27. The process according to claim 26, wherein ethanol is obtained as microbial metabolite.

28. A glucose product which can be obtained by a process as defined in any of claims 1 to 25.

29. A lignin product which can be obtained by a process as defined in any of claims 1 to 25.
